(19) United States
(12) Patent Application Publication
(10) Pub. No.: US 2008/0168607 A1
(43) Pub. Date: Jul. 17, 2008

(54) Unsymmetrical Diazo Compounds, Compositions Comprising Same, Dyeing Method and Device Comprising Said Compositions

(76) Inventors: Herve David, La Varenne Saint Hilaire (FR); Andrew Greaves, Montevrain (FR); Nicolas Daubresse, La Celles St. Cloud (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/793,013

(22) PCT Filed: Dec. 15, 2005
(86) PCT No.: PCT/EP2005/014208

§ 371 Date: Nov. 8, 2007

Related U.S. Application Data
(60) Provisional application No. 60/646,976, filed on Jan. 27, 2005.

(30) Foreign Application Priority Data
Dec. 15, 2004 (FR) 0453001

(51) Int. Cl. A61K 8/00

(52) U.S. Cl. 8/405

(57) ABSTRACT

The invention concerns symmetric diazo cationic compounds of formula (I), their resonance forms, as well as their acid-addition salts and their solvates, wherein W₁, identical represent a hydrogen atom, a halogen atom, a —NR₅R₆, OR₇, —NR₁-Ph-NR₅R₆, —NR₄-Ph-OR₇, —O-Ph-OR₇, —O-Ph-NR₅R₆ group; L represents a cationic of non-cationic linker arm. The invention also concerns dyeing compositions comprising in a suitable medium for dyeing keratinous fibers, such compounds as direct dyeing agent, as well as a method for dyeing keratinous fibers using said composition and a multiple-compartment device.

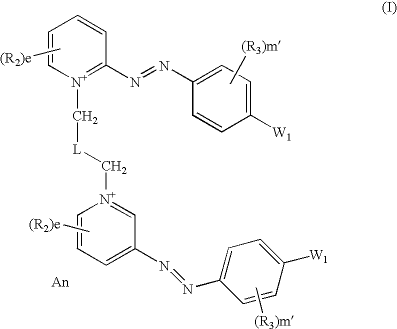

A subject matter of the present invention is specific asymmetric diazo cationic compounds comprising a cationic or noncationic connecting arm, dyeing compositions comprising, in a medium appropriate for the dyeing of keratinous fibers, such compounds as direct dye, and also a process for coloring keratinous fibers employing this composition and a multi-compartment device.

It is known to dye keratinous fibers and in particular human keratinous fibers, such as the hair, with dyeing compositions comprising direct dyes. These compounds are colored and coloring molecules having an affinity for the fibers. It is known, for example, to use direct dyes of the nitrobenzene type, anthraquinone dyes, nitropyridines or dyes of the azo, xanthene, acridine, azine or triarylmethane type.

Usually, these dyes are applied to the fibers, optionally in the presence of an oxidizing agent if it is desired to obtain a simultaneous effect of lightening of the fibers. Once the leave-in time has passed, the fibers are rinsed, optionally washed and dried.

The colorings which result from the use of direct dyes are temporary or semipermanent colorings as the nature of the interactions which bond the direct dyes to the keratinous fiber and their desorption from the surface and/or from the core of the fiber are responsible for their low relative dyeing power and for their poor relative resistance to washing operations or to perspiration.

It is known, in application EP 1 377 263, to employ specific diazo cationic direct dyes comprising two cationic heterocyclic groups. These compounds, while they represent an advance in the field, give dyeing results which even so can still be improved.

Within the meaning of the present invention and unless otherwise indicated:
an alkyl(ene) radical or the alkyl(ene) part of a radical is linear or branched,
an alkyl(ene) radical or the alkyl(ene) part of a radical is said to be “substituted” when it comprises at least one substituent chosen from the groups:
   hydroxyl,
   C₁-C₄ alkoxy or C₂-C₄ (poly)hydroxyalkoxy,
   amino or amino substituted by one or two identical or different C₁-C₄ alkyl groups which optionally carry at least one hydroxyl or C₁-C₂ alkoxy group, it being possible for said alkyl radicals to form, with the nitrogen atom to which they are attached, a saturated or unsaturated, optionally aromatic, optionally substituted, 5- or 7-membered heterocycle optionally comprising at least one other heteroatom different from or the same as nitrogen,
   an alkylcarbonylamino (R′CO—NR—) radical in which the R radical is a hydrogen atom or a C₁-C₄ alkyl radical,
   an alkylsulfonyl (R—SO₂—) radical in which the R radical represents a C₁-C₄ alkyl radical,
   an alkylsulfinyl (R—SO—) radical in which the R radical represents a C₁-C₄ alkyl radial,
   an alkylcarbonyl (R—CO—) radical in which the R radical represents a C₁-C₄ alkyl radical,
a saturated or unsaturated and aromatic or non-aromatic (hetero)cyclic radical or the saturated or unsaturated and aromatic or nonaromatic (hetero)cyclic part of a radical is said to be “substituted” when it comprises at least one substituent, preferably carried by a carbon atom, chosen from:
   a C₁-C₁₆, preferably C₁-C₈, alkyl radical which is optionally substituted;
   a halogen atom, such as chlorine, fluorine or bromine;
   a hydroxyl group;
   a C₁-C₄ alkoxy radical or a C₂-C₄ (poly)hydroxy-alkoxy radical;
   an amino radical;
   an amino radical substituted by one or two identical or different C₁-C₄ alkyl radicals which optionally carry at least one hydroxyl or amino or mono- or di(C₁-C₄)alkylamino or C₁-C₂ alkoxy group; it being possible for the two alkyl radicals to form, with a nitrogen atom to which they are attached, a heterocycle including 1 to 3 heteroatoms, preferably 1 or 2 heteroatoms, chosen from N, O or S, preferably N, the heterocycle comprising from 5 to 7 ring members and being saturated or unsaturated and aromatic or nonaromatic and optionally substituted;
   an alkylcarbonylamino (R′CO—NR—) radical in which the R radical is a hydrogen atom or a C₁-C₄ alkyl radical and the R′ radical is a C₁-C₂ alkyl radical;
   an aminocarbonyl ((R)₂N—CO—) radical in which the R radicals, which are identical or different, represent a hydrogen atom or a C₁-C₄ alkyl radical;
   an alkylsulfonylamino (R′SO₂—NR—) radical in which the R radical represents a hydrogen atom or a C₁-C₄ alkyl radical and the R′ radical represents a C₁-C₄ alkyl radical or a phenyl radical;
   an aminosulfonyl ((R)₂N—SO₂—) radical in which the R radicals, which are identical or different, represent a hydrogen atom or a C₁-C₄ alkyl radical.

The compounds according to the present invention are said to be asymmetric when there does not exist a plane of symmetry perpendicular to the connecting arm L. In other words, the two formula members on either side of the connecting arm L are different. More specifically, they are different when their substituents are different in their natures and/or their positions in the molecule.

The aim of the present invention is to provide direct dyes which do not exhibit the disadvantages of the existing direct dyes.

A subject matter of the present invention is thus cationic asymmetric diazo compounds of following formula (I), their resonance forms, and also their addition salts with an acid and/or their solvates:

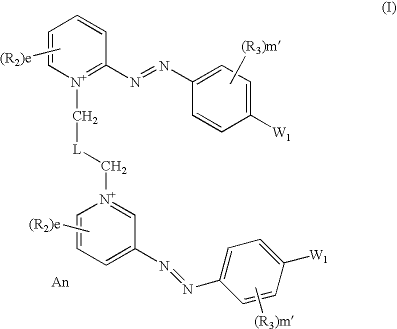

in which formula:
the R₂ radicals, which are identical or different, represent, independently of one another:
an optionally substituted C₁-C₁₆ alkyl radical which is optionally interrupted by one or more heteroatoms and/or by one or more groups comprising at least one heteroatom preferably chosen from oxygen, nitrogen, sulfur, —CO—, —SO₂— or their combinations; said alkyl radical being in addition optionally substituted by one or more identical or different groups chosen from thiol (—SH), C₁-C₄ thioalkyl, (C₁-C₄)alkylsulfinyl or (C₁-C₄)alkylsulfonyl groups;
a hydroxyl group;
a C₁-C₄ alkoxy group;
a C₂-C₄ (poly)hydroxyalkoxy group;
an alkoxycarbonyl (RO—CO—) group in which R represents a C₁-C₄ alkyl radical;
an alkylcarbonyloxy (RCO—O—) radical in which R represents a C₁-C₄ alkyl radical;
an alkylcarbonyl (R—CO—) radical in which R represents a C₁-C₄ alkyl radical;
an amino group;
an amino group substituted by one or two identical or different C₁-C₄ alkyl radicals which optionally carry at least one hydroxyl group, it being possible for the two alkyl radicals optionally to form, with the nitrogen atom to which they are attached, a heterocycle which includes 1 to 3 heteroatoms, preferably 1 or 2 heteroatoms, chosen from N, O or S, preferably N, which comprises from 5 to 7 ring members, which is saturated or unsaturated, which is aromatic or nonaromatic and which is optionally substituted;
an alkylcarbonylamino (RCO—NR′—) group in which the R radical represents a C₁-C₄ alkyl radical and the R′ radical represents a hydrogen or a C₁-C₄ alkyl radical;
an aminocarbonyl ((R)₂N—CO—) group in which the R radicals represent, independently of one another, a hydrogen atom or a C₁-C₄ alkyl radical;
a ureido (N(R)₂—CO—NR′—) group in which the R and R′ radicals represent, independently of one another, a hydrogen atom or a C₁-C₄ alkyl radical;
an aminosulfonyl ((R)₂N—SO₂—) group in which the R radicals represent, independently of one another, a hydrogen atom or a C₁-C₄ alkyl radical;
an alkylsulfonylamino (RSO₂—NR′—) group in which R represents a C₁-C₄ alkyl radical and R′ represents a hydrogen atom or a C₁-C₄ alkyl radical;
an optionally substituted aryl radical;
an optionally substituted aryl(C₁-C₄)alkyl radical;
an alkylsulfinyl (R—SO—) group in which R represents a C₁-C₄ radical;
an alkylsulfonyl (R—SO₂—) group in which R represents a C₁-C₄ radical;
a nitro group;
a cyano group;
a halogen atom, preferably chlorine or fluorine;
a thiol (HS—) group;
an alkylthio (RS—) group in which the R radical represents an optionally substituted C₁-C₄ alkyl radical;
when e is equal to 2, the two R₂ radicals can optionally form, with the carbon atoms to which they are attached, a 5- or 6-membered, preferably 6-membered, aromatic or nonaromatic, secondary ring optionally substituted by one or more identical or different groups chosen from hydroxyl, C₁-C₄ alkyl, C₁-C₄ alkoxy, C₂-C₄ (poly)hydroxyalkoxy, amino or amino substituted by one or two identical or different C₁-C₄ alkyl radicals which optionally carry at least one hydroxyl group;

e is an integer having a value from 0 to 4; when e is less than 4, the unsubstituted carbon atom or atoms of the heterocycle carry a hydrogen atom;

the R₃ radicals, which are identical or different, represent, independently of one another:
an optionally substituted C₁-C₁₆ alkyl radical which is optionally interrupted by one or more heteroatoms or by one or more groups comprising at least one heteroatom preferably chosen from oxygen, nitrogen, sulfur, —CO—, —SO₂— or their combinations;
a hydroxyl group;
a C₁-C₄ alkoxy group;
a C₂-C₄ (poly)hydroxyalkoxy group;
an alkoxycarbonyl (RO—CO—) group in which R represents a C₁-C₄ alkyl radical;
an alkylcarbonyloxy (RCO—O—) radical in which R represents a C₁-C₄ alkyl radical;
an alkylcarbonyl (R—CO—) radical in which R represents a C₁-C₄ alkyl radical;
an amino group;
an amino group substituted by one or two identical or different C₁-C₄ alkyl radicals which optionally carry at least one hydroxyl group, it being possible for the two alkyl radicals optionally to form, with the nitrogen atom to which they are attached, a heterocycle which includes 1 to 3 heteroatoms, preferably 1 or 2 heteroatoms, chosen from N, O or S, preferably N, which comprises from 5 to 7 ring members, which are saturated or unsaturated, which is aromatic or nonaromatic and which is optionally substituted;
an alkylcarbonylamino (RCO—NR′—) group in which the R radical represents a C₁-C₄ alkyl radical and the R′ radical represents a hydrogen atom or a C₁-C₄ alkyl radical;
an aminocarbonyl ((R)₂N—CO—) group in which the R radicals represent, independently of one another, a hydrogen atom or a C₁-C₄ alkyl radical;
a ureido (N(R)₂—CO—NR′—) group in which the R and R′ radicals represent, independently of one another, a hydrogen atom or a C₁-C₄ alkyl radical;
an aminosulfonyl ((R)₂N—SO₂—) group in which the R radicals represent, independently of one another, a hydrogen atom or a C₁-C₄ alkyl radical;
an alkylsulfonylamino (RSO₂—NR′—) group in which the R and R′ radicals represent, independently of one another, a hydrogen atom or a C₁-C₄ alkyl radical;
a thiol (HS—) group;
an alkylthio (RS—) group in which the R radical represents a C₁-C₄ alkyl radical;
an alkylsulfinyl (R—SO—) group in which R represents a C₁-C₄ alkyl radical;
an alkylsulfonyl (R—SO₂—) group in which R represents a C₁-C₄ alkyl radical;
a nitro group;
a cyano group;
a halogen atom, preferably chlorine or fluorine;
when m′ is greater than or equal to 2, two adjacent R₃ radicals can form, with the carbon atoms to which they are attached, a 6-membered, aromatic or nonaromatic, secondary ring optionally substituted by one or more identical or different groups chosen from the following groups: hydroxyl, C₁-C₄ alkyl, C₁-C₄ alkoxy, C₂-C₄ (poly)hydroxy-alkoxy, C₁-C₄ alkylcarbonylamino, amino or amino substituted by one or two identical or different C₁-C₄ alkyl radicals which optionally carry at least one hydroxyl group;

m′ is an integer having a value from 0 to 4; when m′ is less than 4, then the unsubstituted carbon atom or atoms of the aromatic ring carry a hydrogen atom;

W₁, which are identical or different, represent, independently of one another:
a hydrogen atom;
a halogen atom chosen from bromine, chlorine or fluorine, preferably chlorine and fluorine;
an —NR₅R₆, —OR₇, —NR₄-Ph-NR₅R₆, —NR₄-Ph-OR₇, —O-Ph-OR₇ or —O-Ph-NR₅R₆ group; with:
   R₄ and R₇, which are identical or different, representing a hydrogen atom, an optionally substituted C₁-C₂₀, preferably C₁-C₁₆, alkyl radical, an optionally substituted ar(C₁-C₃)-alkyl radical or an optionally substituted phenyl radical;
   R₅ and R₆, which are identical or different, representing a hydrogen atom, an optionally substituted C₁-C₂₀, preferably C₁-C₁₆, alkyl radical, an optionally substituted phenyl radical, an optionally substituted ar(C₁-C₃)-alkyl radical or an alkylcarbonyl (R—CO—) radical in which R is a C₁-C₄ alkyl radical;
   R₅ and R₆ being able optionally to form, with the nitrogen atom to which they are attached, a heterocycle which includes 1 to 3 heteroatoms, preferably 1 or 2 heteroatoms, chosen from N, O or S, preferably N, which comprises from 5 to 7 ring members, which is saturated or unsaturated, which is aromatic or nonaromatic and which is optionally substituted;
   R₅ and R₆ being able to form, with the carbon atom of the aromatic ring adjacent to that to which —NR₅R₆ is attached, a saturated 5- or 6-membered heterocycle;
   Ph representing an optionally substituted phenyl radical;

L is a cationic or noncationic connecting arm;

the electrical neutrality of the compound of formula (I) being provided by one or more identical or different anions An which are cosmetically acceptable.

A subject matter of the present invention is likewise dyeing compositions comprising, in a medium appropriate for the dyeing of keratinous fibers, such compounds or their addition salts with an acid as direct dyes.

The invention furthermore relates to a process for coloring keratinous fibers which consists in bringing a composition according to the invention into contact with said dry or wet fibers for a period of time sufficient to obtain the desired effect.

Finally a subject matter of the invention is a multi-compartment device comprising, in a first compartment, the composition according to the invention and, in a second compartment, an oxidizing composition.

It has been found that the compounds of formula (I) as defined above exhibit good persistence with regard to external agents, such as in particular shampoos, this being the case even when the keratinous fiber is sensitized. Furthermore, these compounds exhibit improved dyeing properties, such as chromaticity or coloring power, and low selectivity, that is to say that the compounds of the invention make it possible to obtain colorings which are more uniform between the tips and the roots of the individual hairs.

However, other characteristics and advantages of the present invention will become more clearly apparent on reading the description and examples which will be presented.

In what follows and unless otherwise indicated, the limits delimiting a range of values are included in this range.

As indicated above, a first subject matter of the invention is compounds corresponding to the above-mentioned formula (I) and their salts and/or solvates.

Preferably, the compounds of formula (I) according to the present invention are such that the R₂ radicals, which are identical or different, preferably represent, independently of one another:
a halogen atom chosen from chlorine or fluorine;
a C₁-C₄ alkyl radical optionally substituted by one or more identical or different radicals chosen from hydroxyl, C₁-C₂ alkoxy, C₂-C₄ (poly)hydroxy-alkoxy, amino, (di)(C₁-C₂)alkylamino, thiol (—SH), (C₁-C₄)alkylsulfinyl, (C₁-C₄)alkylsulfonyl or thio (C₁-C₄)alkyl radicals;
a phenyl radical optionally substituted by one or more radicals chosen from hydroxyl, C₁-C₂ alkoxy, C₂-C₄ (poly)hydroxyalkoxy, amino or (di)(C₁-C₂)alkylamino radicals or a halogen atom, such as chlorine or fluorine;
a C₁-C₄ alkoxy radical;
a (C₁-C₄)alkylsulfonylamino radical;
a C₂-C₄ (poly)hydroxyalkoxy radical;
an amino radical;
a (di)(C₁-C₂)alkylamino radical;
a C₂-C₄ (poly)hydroxyalkylamino radical;
an alkylsulfonylamino (RSO₂N—) radical in which the R radical represents a C₁-C₄ alkyl radical;
an aminosulfonyl ((R)₂NSO₂—) radical in which the R radicals represent, independently of one another, a hydrogen atom or a C₁-C₄ alkyl radical;
an alkylthio (RS—) radical in which the R radical represents a C₁-C₄ alkyl radical;
an alkylsulfinyl (RSO—) radical in which the R radical represents a C₁-C₄ alkyl radical;
an alkylsulfonyl (R—SO₂—) radical in which the R radical represents a C₁-C₄ alkyl radical;
an alkylcarbonylamino (RCONR′—) radical in which the R radical represents a hydrogen atom or a C₁-C₄ alkyl radical and the R′ radical represents a hydrogen atom or a C₁-C₄ alkyl radical.

According to a particularly preferred embodiment, the R₂ radicals, which are identical or different, preferably represent, independently of one another, a methyl, ethyl, 2-hydroxyethyl, 2-methoxyethyl, methyl-sulfonyl (CH₃SO₂—), methylcarbonylamino (CH₃CONH—), hydroxyl, amino, methylamino, dimethylamino, 2-hydroxy-ethylamino, methoxy, ethoxy or phenyl radical.

According to a second preferred alternative form, two R₂ radicals can optionally form, with the carbon atoms to which they are attached, a 6-membered aromatic secondary ring optionally substituted by one or more identical or different groups chosen from hydroxyl, C₁-C₄ alkyl, C₁-C₄ alkoxy, amino or amino substituted by one or two identical or different C₁-C₄ alkyl radicals which optionally carry at least one hydroxyl or methylcarbonylamino group.

In accordance with this second alternative form, two R₂ radicals can optionally form, with the carbon atoms to which they are attached, a 6-membered aromatic secondary ring optionally substituted by one or more identical or different radicals chosen from hydroxyl, methoxy, ethoxy, amino, acylamino, 2-hydroxyethylamino, dimethylamino, (di)(2-hydroxyethyl)amino, methyl-carbonylamino.

According to a very particularly advantageous embodiment, the coefficient e is equal to 0.

As relates more particularly to the R₃ radicals, the latter, which are identical or different, more particularly represent, independently of one another:
a C₁-C₁₆, preferably C₁-C₈, alkyl radical which is optionally substituted;
a halogen atom, such as chlorine or fluorine;
a hydroxyl group;
a C₁-C₂ alkoxy radical;
a C₂-C₄ (poly)hydroxyalkoxy radical;
an amino radical;
an amino radical substituted by one or two identical or different C₁-C₄ alkyl radicals which optionally carry at least one hydroxyl group or at least one C₁-C₄ alkoxy radical, it being possible for the two alkyl radicals to form, with the nitrogen atom to which they are attached, a heterocycle including 1 to 3 heteroatoms, preferably 1 or 2 heteroatoms, chosen from N, O or S, preferably N, the heterocycle comprising from 5 to 7 ring members, being saturated or unsaturated, being aromatic or nonaromatic and optionally being substituted;
an alkylcarbonylamino (RCO—NR′—) radical in which the R radical represents a C₁-C₄ alkyl radical and the R′ radical represents a hydrogen or a C₁-C₄ alkyl radical;
an alkylsulfonylamino (R′SO₂—NR—) radical in which the R radical represents a hydrogen atom or a C₁-C₄ alkyl radical and the R′ radical represents a C₁-C₄ alkyl radical;
an aminosulfonyl ((R)₂N—SO₂—) radical in which the R radicals, which are identical or different, represent a hydrogen atom or a C₁-C₄ alkyl radical;
an alkylthio (RS—) radical in which the R radical represents a C₁-C₄ alkyl radical;
an alkylsulfonyl (R—SO₂—) radical in which the R radical represents a C₁-C₄ alkyl radical.

More preferably, said identical or different R₃ radicals represent, independently of one another:
a C₁-C₄ alkyl radical optionally substituted by one or more identical or different radicals chosen from the following radicals: hydroxyl, C₁-C₂ alkyl-carbonylamino or amino substituted by two identical or different C₁-C₂ alkyl radicals which optionally carry at least one hydroxyl group or a C₁-C₂ alkoxy radical; these two alkyl radicals can optionally form, with the nitrogen atom to which they are attached, a saturated or unsaturated, optionally aromatic, 5- or 6-membered heterocycle preferably chosen from pyrrolidine, piperazine, homopiperazine, pyrrole, imidazole or pyrazole;
a C₂-C₄ hydroxyalkoxy radical;
a halogen chosen from chlorine or fluorine;
an amino radical;
an amino radical substituted by one or two identical or different C₁-C₂ alkyl radicals which optionally carry at least one hydroxyl group;
a methylcarbonylamino radical;
a methylsulfonylamino radical;
a hydroxyl radical;
a C₁-C₂ alkoxy radical;
a methylsulfonyl radical.

According to this alternative form, the R₃ radicals preferably represent, independently of one another:
a methyl, ethyl, propyl, 2-hydroxyethyl, methoxy, ethoxy, 2-hydroxyethyloxy, 3-hydroxypropyloxy or 2-methoxyethyl radical;
a methylsulfonylamino radical;
an amino, methylamino, dimethylamino or 2-hydroxy-ethylamino radical;
a methylcarbonylamino radical;
a hydroxyl radical;
a chlorine atom;
a methylsulfonyl radical.

According to a second preferred alternative form, when the coefficient m′ is greater than or equal to 2, then two adjacent R₃ radicals can form, with the carbon atoms to which they are attached, a 6-membered aromatic secondary ring optionally substituted by one or more identical or different groups chosen from the following groups: hydroxyl, —NR₄-Ph, —NR₄-Ph-NR₅R₆, —NR₄-Ph-OR₇, C₁-C₄ alkyl, C₁-C₄ alkoxy, C₂-C₄ (poly)hydroxyalkoxy, C₁-C₄ alkylcarbonylamino, amino or amino substituted by one or two identical or different C₁-C₄ alkyl radicals which optionally carry at least one hydroxyl group.

According to this second alternative form and more advantageously still, two adjacent R₃ radicals can form, with the carbon atoms to which they are attached, a 6-membered aromatic secondary ring optionally substituted by one or more identical or different groups chosen from the hydroxyl, methoxy, ethoxy, 2-hydroxyethyloxy, amino, methylcarbonylamino, (di)(2-hydroxyethyl)amino, —NH-Ph, —NH-Ph-NH₂, —NH-Ph-NHCOCH₃, —NH-Ph-OH or —NH-Ph-OCH₃ groups.

As relates to the R₄ and R₇ radicals, the latter represent:
a hydrogen atom;
a C₁-C₆ alkyl radical which is optionally substituted, preferably by at least one hydroxyl or C₁-C₂ alkoxy group;
an aryl or arylalkyl radical, such as phenyl or benzyl, the aryl part optionally being substituted by one or more identical or different radicals chosen from chlorine, amino, hydroxyl, C₁-C₂ alkoxy or amino mono- or disubstituted by one or two identical or different C₁-C₄ alkyl radicals which optionally carry at least one hydroxyl group.

In accordance with a preferred embodiment of the invention, the R₄ and R₇ radicals represent:
a hydrogen atom;
an optionally substituted C₁-C₃ alkyl radical, such as methyl, ethyl, 2-hydroxyethyl or 2-methoxyethyl;
a phenyl radical optionally substituted by one or more radicals chosen from the following radicals: hydroxyl, C₁-C₂ alkoxy, amino or amino substituted by one or more C₁-C₄ groups which optionally carry at least one hydroxyl group.

Preferably, the R₄ and R₇ radicals represent:
a hydrogen atom;
a methyl, ethyl or 2-hydroxyethyl radical;
a phenyl radical optionally substituted by a hydroxyl, methoxy, amino, (di)methylamino or (di)(2-hydroxyethyl)amino radical.

As relates to the identical or different R₅ and R₆ radicals, the latter more particularly represent, independently of one another:
a hydrogen atom;
an alkylcarbonyl (R—CO—) radical in which R represents an optionally substituted C₁-C₄ alkyl radical;
a C₁-C₆ alkyl radical which is optionally substituted, preferably by one or more identical or different groups chosen from hydroxyl, C₁-C₂ alkoxy, amino or (di)(C₁-C₄)alkylamino; the alkyl radical can, in addition, be substituted by at least one C₁-C₄ alkylsulfonyl group, at least one C₁-C₄ alkylsulfinyl group or at least one C₁-C₄ alkylcarbonyl group;
an aryl or arylalkyl radical, such as phenyl or benzyl, the aryl part optionally being substituted by one or more identical or different groups chosen from chlorine, amino, hydroxyl, C₁-C₄ alkoxy or amino mono- or disubstituted by one or two identical or different C₁-C₄ alkyl radicals which optionally carry at least one hydroxyl group.

In accordance with a preferred embodiment of the invention, the identical or different R₅ and R₆ radicals advantageously represent, independently of one another:
a hydrogen atom;
a methylcarbonyl, ethylcarbonyl or propylcarbonyl radical;
an optionally substituted C₁-C₃ alkyl radical, such as methyl, ethyl, 2-hydroxyethyl or 2-methoxy-ethyl;
a phenyl radical optionally substituted by one or more radicals chosen from the following radicals: hydroxyl, C₁-C₂ alkoxy, amino or amino substituted by one or more C₁-C₄ groups which optionally carry at least one hydroxyl group.

More preferably still, the identical or different R₅ and R₆ radicals represent, independently of one another:
a hydrogen atom;
a methyl, ethyl or 2-hydroxyethyl radical;
a methylcarbonyl, ethylcarbonyl or propylcarbonyl radical;
a phenyl radical optionally substituted by a hydroxyl, methoxy, amino, (di)methylamino or (di)(2-hydroxyethyl)amino radical.

It should be noted that, according to a specific embodiment of the invention, the R₅ and R₆ radicals form, together with the nitrogen atom to which they are attached, a heterocycle which includes 1 to 3 hetero-atoms, preferably 1 or 2 heteroatoms, chosen from N, O or S, preferably N, which comprises from 5 to 7 ring members, which is saturated or unsaturated, which is aromatic or nonaromatic and which is optionally substituted.

Advantageously, the heterocycle comprising from 5 to 7 ring members is chosen from the following heterocycles: piperidine, 2-(2-hydroxyethyl)piperidine, 4-(amino-methyl)piperidine, 4-(2-hydroxyethyl)piperidine, 4-(dimethylamino)piperidine, piperazine, 1-methyl-piperazine, 1-(2-hydroxyethyl)piperazine, 1-(2-amino-ethyl)piperazine, 1-hydroxyethylethoxy piperazine, homopiperazine, 1-methyl-1,4-perhydrodiazepine, pyrrole, 1,4-dimethylpyrrole, 1-methyl-4-ethylpyrrole or 1-methyl-4-propylpyrrole.

Preferably, the heterocycle comprising from 5 to 7 ring members represents a heterocycle of the following types: piperidine, piperazine, homopiperazine, pyrrole, imidazole or pyrazole optionally substituted by one or more methyl, hydroxyl, amino or (di)methylamino radicals.

According to a third alternative form, the R₅ and R₆ radicals form, with the carbon atoms of the aromatic ring, optionally substituted by a hydroxyl, adjacent to that to which —NR₅R₆ is attached, a saturated 5- or 6-membered heterocycle.

For example, the —NR₅R₆ group with the aromatic nucleus optionally substituted by a hydroxyl can correspond to the following compounds:

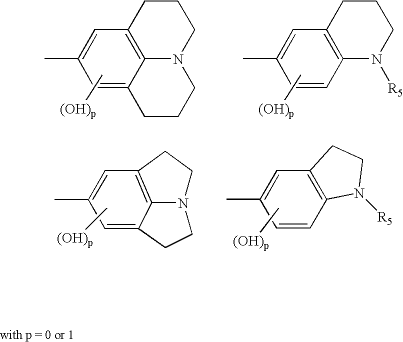

In a first alternative form, L is a noncationic connecting arm.

According to this alternative form, L, a noncationic connecting arm connecting the two different azo chromo-phores, represents:
a covalent bond;
a C₁-C₄₀, preferably C₁-C₂₀, alkyl radical which is optionally substituted and optionally interrupted by a saturated or unsaturated, aromatic or nonaromatic, 3- to 7-membered (hetero)cycle which is optionally substituted and optionally condensed; said alkyl radical optionally being interrupted by one or more heteroatoms or groups comprising at least one heteroatom, preferably oxygen, nitrogen, sulfur, —CO—, —SO₂— or their combinations; the connecting arm L not comprising an azo, nitro, nitroso or peroxo bond;
an optionally substituted phenyl radical.

According to a second alternative form, the connecting arm is cationic.

According to this alternative form, L, a cationic connecting arm connecting the two different azo chromo-phores, represents a C₂-C₄₀ alkyl radical carrying at least one cationic charge, which alkyl radical is optionally substituted and/or optionally interrupted by one or more identical or different, saturated or unsaturated, aromatic or nonaromatic, 3- to 7-membered (hetero)cycles and/or optionally interrupted by one or more heteroatoms or groups comprising at least one heteroatom or their combinations, such as, for example, oxygen, nitrogen, sulfur, a —CO— or —SO₂— group or their combinations; the connecting arm L not comprising an azo, nitro, nitroso or peroxo bond; it being understood that the connecting arm L carries at least one cationic charge.

According to a first specific alternative form, L represents a noncationic connecting arm.

According to this alternative form, mention may be made, as example of connecting arm L of alkyl type which is preferred, of the methylene, ethylene, linear or branched propylene, linear or branched butylene, linear or branched pentylene or linear or branched hexylene radicals, optionally substituted and/or interrupted as indicated above.

These identical or different substituents are preferably chosen from hydroxyl, C₁-C₂ alkoxy, di(C₁-C₂)alkylamino, (C₁-C₄)alkylcarbonyl or (C₁-C₄)alkylsulfonyl.

Mention may be made, as preferred examples of saturated or unsaturated, aromatic or nonaromatic, ring or heterocycle which interrupts the alkyl radical of the connecting arm L, of the phenylene, naphthylene, phenanthrylene, triazinyl, pyrimidinyl, pyridinyl, pyridazinyl, quinoxalinyl or cyclohexyl radicals.

Mention may be made, as examples of connecting arm L, of the methylene, ethylene, linear or branched propylene, linear or branched butylene, linear or branched pentylene or linear or branched hexylene radicals, optionally substituted and/or interrupted as indicated above.

Mention may be made, as examples of saturated or unsaturated, aromatic or nonaromatic, ring or hetero-cycle which interrupts the alkyl radical of the connecting arm L, of the phenylene, naphthylene, phenanthrylene, triazinyl, pyrimidinyl, pyridinyl, pyridazinyl, quinoxalinyl or cyclohexyl radicals.

The following L radicals are more particularly suitable:

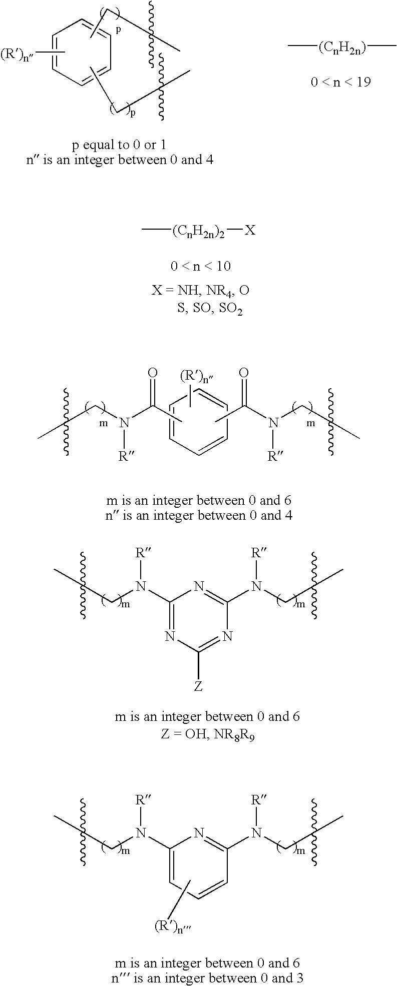

in which formulae:
R′ has the same definition as R₃;
R″, which are identical, represent a hydrogen or a C₁-C₄ alkyl radical;
R₈ and R₉ represent, independently of one another, a hydrogen atom, a C₁-C₈ alkyl radical optionally substituted by one or more identical or different radicals chosen from hydroxyl, C₁-C₂ alkoxy, C₂-C₄ (poly)hydroxyalkoxy, amino or (di)(C₁-C₂)alkylamino or an optionally substituted aryl radical.

Mention may also be made, as examples of L radicals which are preferred, of:

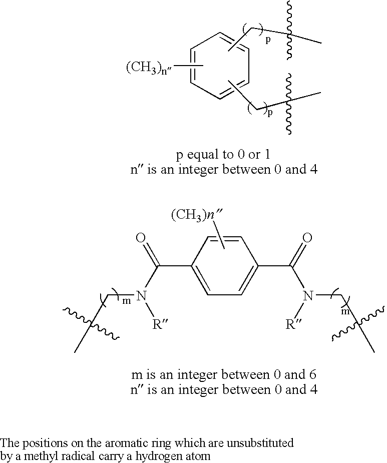

According to a second specific alternative form, L represents a cationic connecting arm.

According to this alternative form, the cationic connecting arm L advantageously represents a C₂-C₂₀ alkyl radical:
1—interrupted by at least one group corresponding to the following formulae:

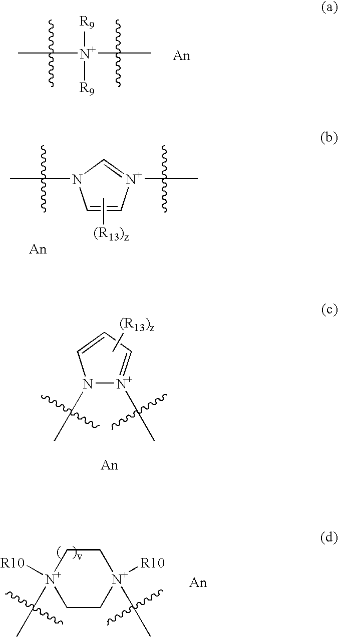

in which:
R₉ and R₁₀ represent, independently of one another, a C₁-C₈ alkyl radical; a C₁-C₆ monohydroxyalkyl radical; a C₂-C₆ polyhydroxyalkyl radical; a (C₁-C₆)alkoxy(C₁-C₆)alkyl radical; an aryl radical, such as optionally substituted phenyl; an aryl-alkyl radical, such as optionally substituted benzyl; a C₁-C₆ aminoalkyl radical; a C₁-C₆ amino-alkyl radical, the amine of which is substituted by one or two identical or different C₁-C₄ alkyl radicals, a (C₁-C₆)alkylsulfonyl radical;
two R₉ radicals can form, together with the nitrogen atom to which they are attached, an optionally substituted, saturated or unsaturated, 5-, 6- or 7-membered ring;
R₁₃, which are identical or different, represent a halogen atom chosen from bromine, chlorine or fluorine, a C₁-C₆ alkyl radical, a C₁-C₆ mono-hydroxyalkyl radical, a C₂-C₆ polyhydroxyalkyl radical, a C₁-C₆ alkoxy radical, a (di)(C₁-C₄)-alkylamino radical, a hydroxycarbonyl radical, a C₁-C₆ alkylcarbonyl radical, a C₁-C₆ thioalkyl radical, a (C₁-C₆)alkylthio radical, a (C₁-C₆)-alkylsulfonyl radical, a benzyl radical which is optionally substituted or a phenyl radical which is optionally substituted by one or more radicals chosen from the methyl, hydroxyl, amino or methoxy radicals;
An is an organic or inorganic anion or a mixture of organic or inorganic anions;
z is an integer between 1 and 3; if z<3, then the unsubstituted carbon atoms carry a hydrogen atom;
v is an integer equal to 1 or 2, preferably equal to 1;
2—optionally interrupted by one or more heteroatoms or groups comprising at least one heteroatom or their combinations, such as, for example, oxygen, nitrogen, sulfur, a —CO— group or an —SO₂— group; with the condition that there is no nitro, nitroso or peroxo group or bond in the connecting arm L;
3—and optionally substituted by one or more radicals chosen from the following radicals: hydroxyl, C₁-C₂ alkoxy, C₂-C₄ (poly)hydroxyalkoxy or amino substituted by one or more linear or branched C₁-C₂ alkyl groups which optionally carry at least one hydroxyl group.

According to a specific embodiment of the formulae (a) and (d), R₉ and R₁₀ separately are preferably chosen from a C₁-C₆ alkyl radical, a C₁-C₄ monohydroxyalkyl radical, a C₂-C₄ polyhydroxyalkyl radical, a (C₁-C₆)-alkoxy(C₂-C₄)alkyl radical or a dimethylamino(C₂-C₆)-alkyl radical.

More preferably still, R₉ and R₁₀ separately represent a methyl, ethyl or 2-hydroxyethyl radical.

According to a specific embodiment of the formulae (b) and (c), R₁₃ represents a halogen atom chosen from chlorine and fluorine, a C₁-C₆ alkyl radical, a C₁-C₄ monohydroxyalkyl radical, a C₁-C₄ alkoxy radical, a hydroxycarbonyl radical, a (C₁-C₆)alkylthio radical or an amino radical disubstituted by a (C₁-C₄)alkyl radical.

According to an even more specific embodiment of the formulae (b) and (c), R₁₃ represents a chlorine atom, a methyl, an ethyl, a 2-hydroxyethyl, a methoxy, a hydroxycarbonyl or a dimethylamino.

According to another even more specific embodiment of the formulae (b) and (c), z is equal to 0.

In the formula (I), An represents an organic or inorganic anion or a mixture of organic or inorganic anions which makes it possible to balance the charge or charges of the compounds of formula (I), for example chosen from a halide, such as chloride, bromide, fluoride or iodide; a hydroxide; a sulfate, a hydrogen-sulfate; an alkyl sulfate for which the alkyl part, which is linear or branched, is a C₁-C₆ part, such as the methyl sulfate or ethyl sulfate ion; carbonates and hydrogencarbonates; salts of carboxylic acids, such as formate, acetate, citrate, tartrate or oxalate; alkyl-sulfonates for which the alkyl part, which is linear or branched, is a C₁-C₆ part, such as the methylsulfonate ion; arylsulfonates for which the aryl part, which is preferably phenyl, is optionally substituted by one or more C₁-C₄ alkyl radicals, such as, for example, 4-toluoylsulfonate; alkylsulfonyls, such as mesylate.

The addition salts with an acid of the compounds of formula (I) can be, by way of example, the addition salts with an organic or inorganic acid, such as hydro-chloric acid, hydrobromic acid, sulfuric acid or alkyl- or phenylsulfonic acids, such as p-toluenesulfonic acid or methylsulfonic acid.

The solvates of the compounds of formula (I) represent the hydrates of such compounds as well as the combination of these compounds with a linear or branched C₁-C₄ alcohol, such as methanol, ethanol, iso-propanol or n-propanol.

In accordance with a preferred embodiment of the invention, the compounds correspond to the following formulae (I′), (I″) or (I′″) as well as to their resonance forms and/or their addition salts with an acid and/or their solvates:

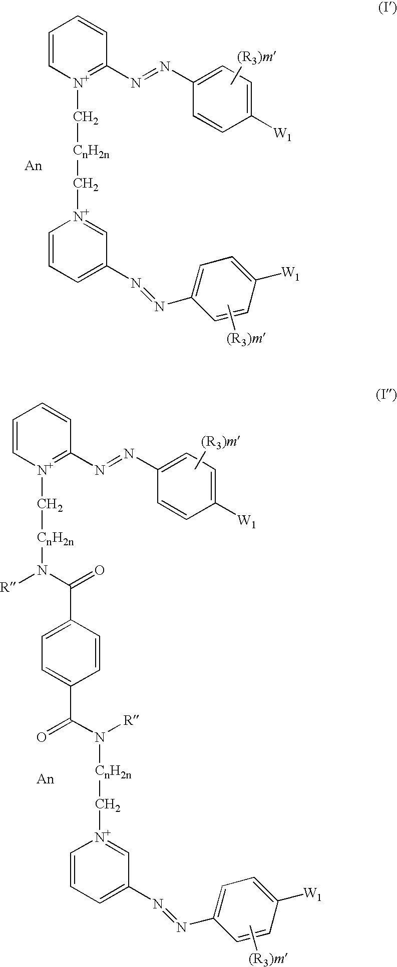

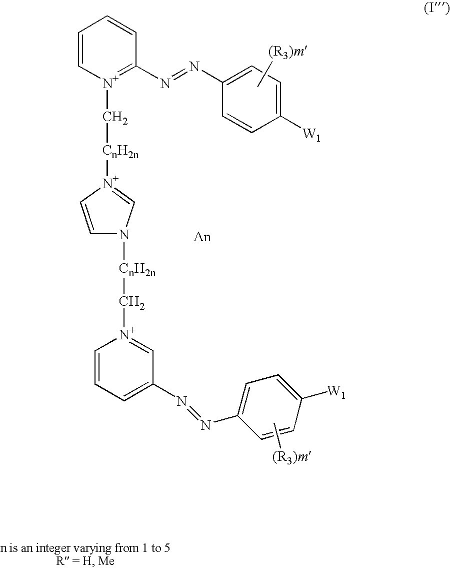

in which R₂, n′ and An have the same meanings as above.

In accordance with a preferred embodiment of the invention, the compounds correspond to one of the following formulae as well as to their resonance forms, their addition salts with an acid and/or their solvates:

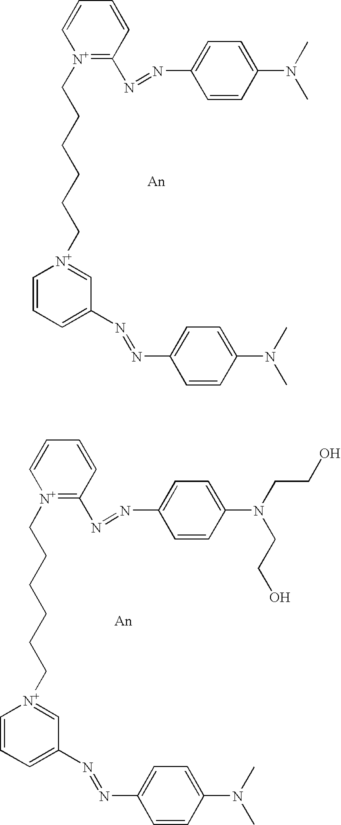

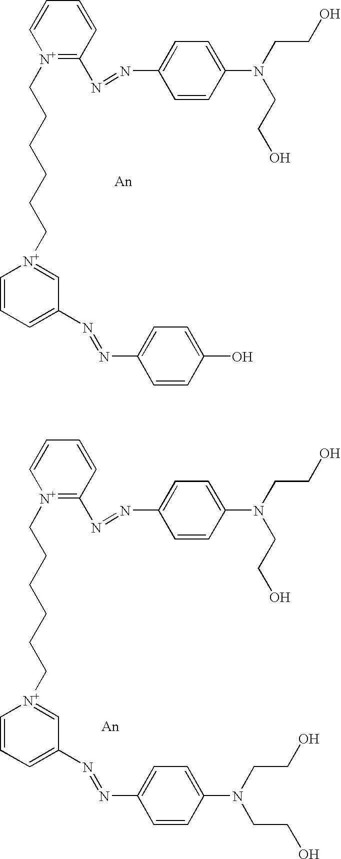

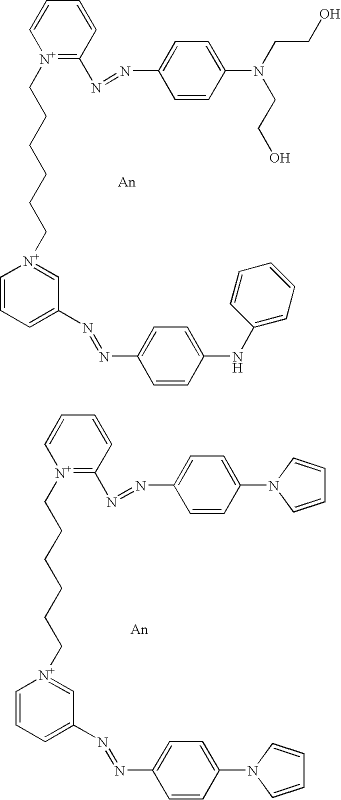

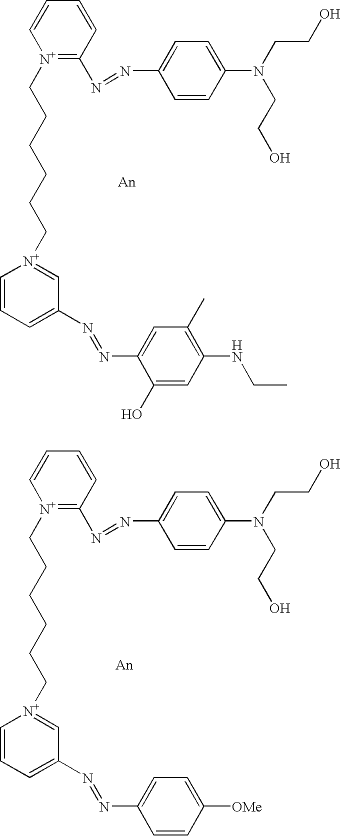

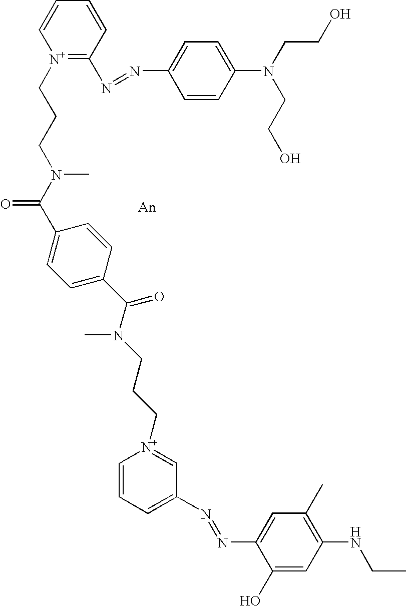

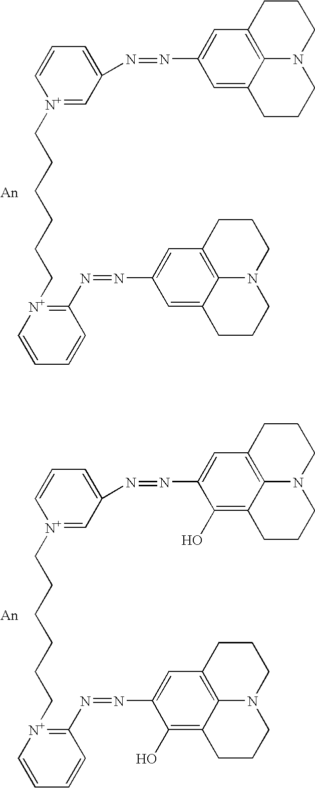

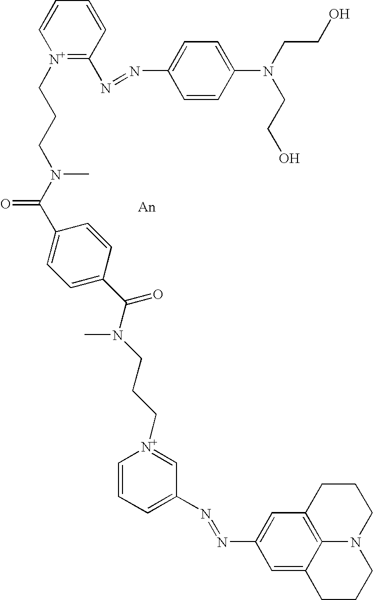

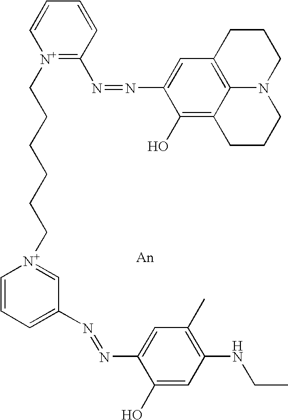

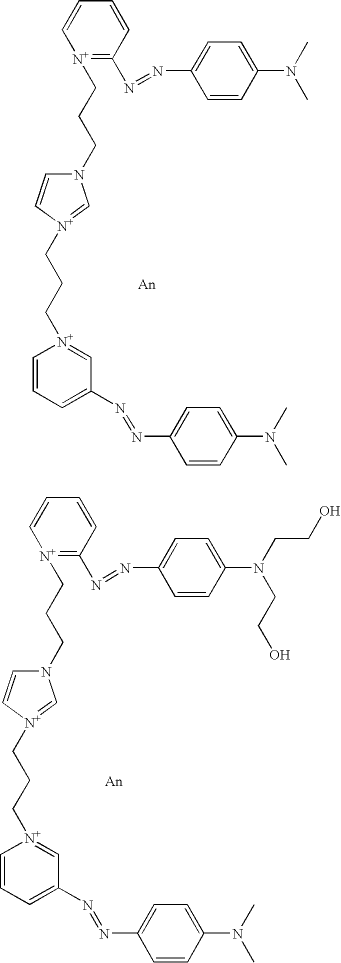

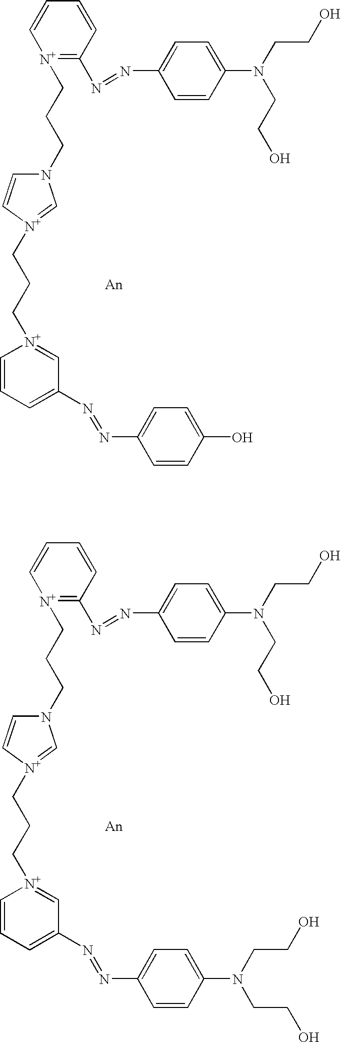

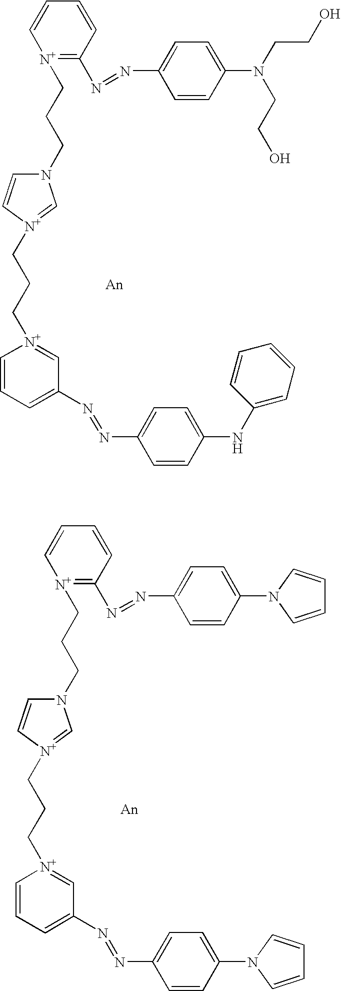

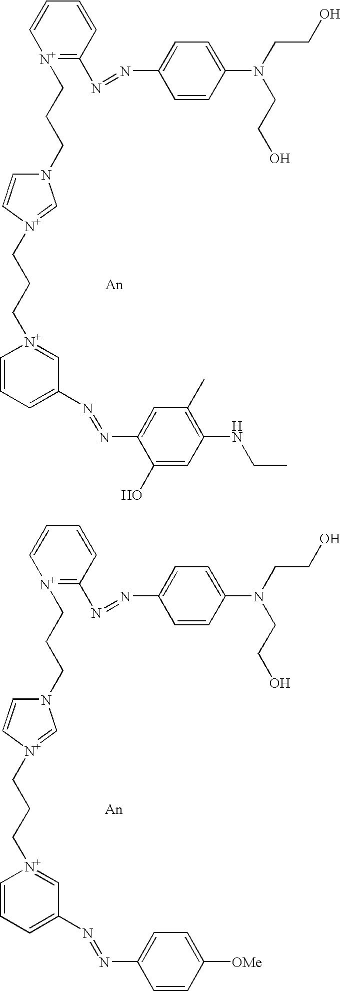

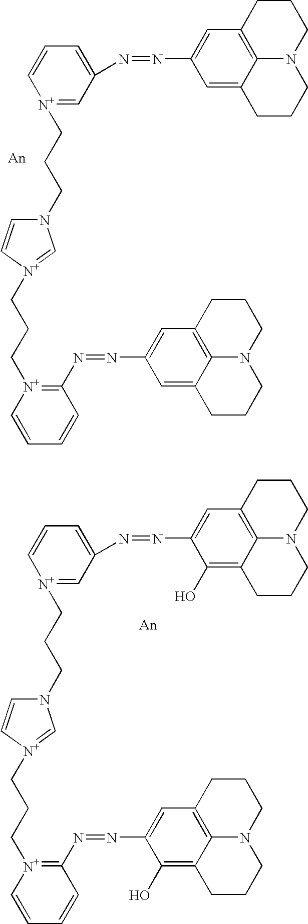

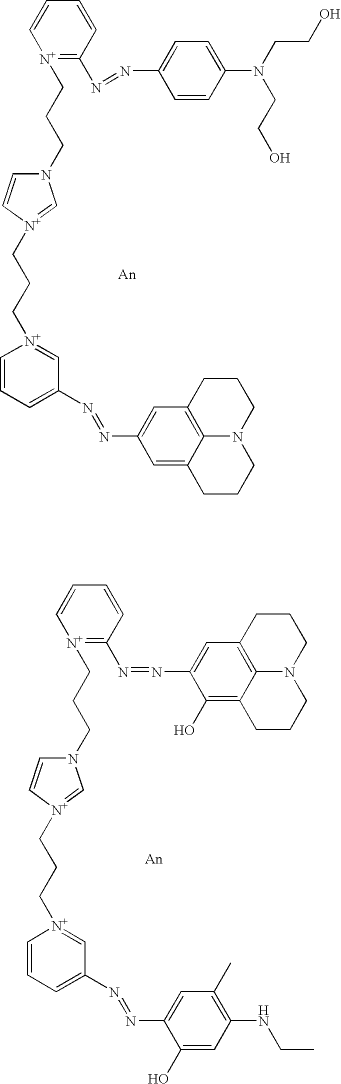

The compounds corresponding to the monoazo entities can in particular be obtained from the preparation processes described, for example, in the documents U.S. Pat. No. 5,708,151, J. Chem. Res., Synop. (1998), (10), 648-649, U.S. Pat. No. 3,151,106, U.S. Pat. No. 5,852,179, Heterocycles, 1987, 26 (2), 313-317, Synth. Commun., 1999, 29(13), 2271-2276, Tetrahedron, 1983, 39 (7), 1091-1101. With regard to the diazo compounds, reference may be made to European application EP 1 377 263 for a description of the synthesis.

Another subject matter of the present invention is composed of a dyeing composition comprising, in a medium appropriate for the dyeing of keratinous fibers, at least one compound of formula (I), its addition salts with an acid and/or its solvates as direct dye.

The total concentration of compound(s) of formula (I) can vary between 0.001 and 20% by weight, with respect to the total weight of the dyeing composition, more particularly between 0.01 and 10% by weight and preferably between 0.05 and 5% by weight.

The dyeing composition according to the invention can furthermore comprise an oxidation base. This oxidation base can be chosen from the oxidation bases conventionally used in oxidation dyeing, for example para-phenylenediamines, bisphenylalkylenediamines, para-aminophenols, ortho-aminophenols and heterocyclic bases.

Mention may more particularly be made, among para-phenylenediamines, by way of example, of para-phenylenediamine, para-toluoylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxy-ethyl)-para-phenylenediamine, 4-N,N-bis(β-hydroxy-ethyl)amino-2-methylaniline, 4-N,N-bis(β-hydroxyethyl)-amino-2-chloroaniline, 2-(β-hydroxyethyl)-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylene-diamine, N,N-dimethyl-3-methyl-para-phenylenediamine, N,N-(ethyl, β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-(β-hydroxyethyloxy)-para-phenylene-diamine, 2-(β-acetylaminoethyloxy)-para-phenylene-diamine, N-(β-methoxyethyl)-para-phenylenediamine, 4-aminophenylpyrrolidine, 2-thienyl-para-phenylene-diamine, 2-(β-hydroxyethylamino)-5-aminotoluene and their addition salts with an acid.

Among the para-phenylenediamines mentioned above, para-phenylenediamine, para-toluoylenediamine, 2-isopropyl-para-phenylenediamine, 2-(β-hydroxyethyl)-para-phenylenediamine, 2-(β-hydroxyethyloxy)-para-phenylene-diamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-(β-acetylaminoethyloxy)-para-phenylenediamine and their addition salts with an acid are particularly preferred.

Mention may be made, among bisphenylalkylenediamines, by way of example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis-(β-hydroxyethyl)-N,N′-bis(4-aminophenyl)tetramethylene-diamine, N,N′-bis(4-methylaminophenyl)tetramethylene-diamine, N,N′-bis(ethyl)-N,N′-bis(4′-amino-3′-methyl-phenyl)ethylenediamine, 1,8-bis(2,5-diaminophenoxy)-3,6-dioxaoctane and their addition salts with an acid.

Mention may be made, among para-aminophenols, by way of example, of para-aminophenol, 4-amino-3-methylphenol, 4-amino-3-fluorophenol, 4-amino-3-(hydroxymethyl)-phenol, 4-amino-2-methylphenol, 4-amino-2-(hydroxy-methyl)phenol, 4-amino-2-(methoxymethyl)phenol, 4-amino-2-(aminomethyl)phenol, 4-amino-2-((β-hydroxy-ethyl)aminomethyl)phenol, 4-amino-2-fluorophenol and their addition salts with an acid.

Mention may be made, among ortho-aminophenols, by way of example, of 2-aminophenol, 2-amino-5-methylphenol, 2-amino-6-methylphenol, 5-acetamido-2-aminophenol and their addition salts with an acid.

Mention may be made, among heterocyclic bases, by way of example, of pyridine derivatives, pyrimidine derivatives and pyrazole derivatives.

Mention may be made, among pyridine derivatives, of the compounds disclosed, for example, in patents GB 1 026 978 and GB 1 153 196, such as 2,5-diaminopyridine, 2-[(4-methoxyphenyl)amino]-3-aminopyridine, 2,3-diamino-6-methoxypyridine, 2-[(β-methoxyethyl)amino]-3-amino-6-methoxypyridine, 3,4-diaminopyridine and their addition salts with an acid.

Mention may be made, among pyrimidine derivatives, of the compounds disclosed, for example, in patents DE 2 359 399; JP 88-169 571; JP 05 163 124; EP 0 770 375 or Patent Application WO 96/15765, such as 2,4,5,6-tetraminopyrimidine, 4-hydroxy-2,5,6-triamino-pyrimidine, 2-hydroxy-4,5,6-triaminopyrimidine, 2,4-di-hydroxy-5,6-diaminopyrimidine or 2,5,6-triamino-pyrimidine, and pyrazolopyrimidine derivatives, such as those mentioned in patent application FR-A-2 750 048 and among which may be mentioned pyrazolo[1,5-a]-pyrimidine-3,7-diamine; 2,5-dimethylpyrazolo[1,5-a]-pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-amino-pyrazolo[1,5-a]pyrimidin-7-ylamino)ethanol, 2-(7-amino-pyrazolo[1,5-a]pyrimidin-3-ylamino)ethanol, 2-[(3-aminopyrazolo[1,5-a]pyrimidin-7-yl)(2-hydroxy-ethyl)amino]ethanol, 2-[(7-aminopyrazolo[1,5-a]-pyrimidin-3-yl)(2-hydroxyethyl)amino]ethanol, 5,6-di-methylpyrazolo[1,5-a]pyrimidine-3,7-diamine, 2,6-di-methylpyrazolo[1,5-a]pyrimidine-3,7-diamine, 2,5,N7,N7-tetramethylpyrazolo[1,5-a]pyrimidine-3,7-diamine, 3-amino-5-methyl-7-(imidazolylpropylamino)pyrazolo-[1,5-a]pyrimidine and their addition salts with an acid and their tautomeric forms, when a tautomeric equilibrium exists.

Mention may be made, among pyrazole derivatives, of the compounds disclosed in patents DE 3 843 892 and DE 4 133 957 and patent applications WO 94/08969, WO 94/08970, FR-A-2 733 749 and DE 195 43 988, such as 4,5-diamino-1-methylpyrazole, 4,5-diamino-1-(β-hydroxy-ethyl)pyrazole, 3,4-diaminopyrazole, 4,5-diamino-1-(4′-chlorobenzyl)pyrazole, 4,5-diamino-1,3-dimethyl-pyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-(tert-butyl)-1-methyl-pyrazole, 4,5-diamino-1-(tert-butyl)-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-(hydroxymethyl)pyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-amino-ethyl)amino-1,3-dimethylpyrazole, 3,4,5-triamino-pyrazole, 1-methyl-3,4,5-triaminopyrazole, 3,5-diamino-1-methyl-4-(methylamino)pyrazole, 3,5-diamino-4-(β-hydroxyethyl)amino-1-methylpyrazole and their addition salts with an acid.

The dyeing composition according to the invention can additionally comprise one or more couplers conventionally used for the dyeing of keratinous fibers. Mention may in particular be made, among these couplers, of meta-phenylenediamines, meta-aminophenols, meta-diphenols, naphthalene couplers and heterocyclic couplers.

Mention may be made, by way of example, of 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diamino-benzene, 1,3-bis(2,4-diaminophenoxy)propane, 3-ureido-aniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxy-ethyl)amino-3,4-methylenedioxybenzene, 2,6-bis-(β-hydroxyethylamino)toluene and their addition salts with an acid.

In the dyeing composition of the present invention, the oxidation base or bases are present in a total amount preferably of between 0.001 and 10% by weight of the total weight of the dyeing composition and more preferably of 0.005 to 6% by weight.

The coupler or couplers are generally present in a total amount of between 0.001 and 10% by weight of the total weight of the dyeing composition and more preferably of 0.005 to 6% by weight.

Generally, the addition salts with an acid which can be used in the context of the dyeing compositions of the invention for the oxidation bases and couplers are chosen in particular from those listed in the context of the definition of the compounds of formula (I).

The composition according to the invention can optionally comprise at least one additional direct dye other than the compounds of formula (I). This can be chosen from cationic or nonionic entities.

Mention may be made, as nonlimiting examples, of nitrobenzene dyes, azo, azomethine, methine, tetraazapentamethine, anthraquinone, naphthoquinone, benzoquinone, phenothiazine, indigoid, xanthene, phenanthridine or phthalocyanine dyes, those derived from triarylmethane and natural dyes, alone or as mixtures.

It can, for example, be chosen from the following red or orangey nitrobenzene dyes:
1-hydroxy-3-nitro-4-[N-(γ-hydroxypropyl)amino]-benzene,
N-(β-hydroxyethyl)amino-3-nitro-4-aminobenzene,
1-amino-3-methyl-4-N-(β-hydroxyethyl)amino-6-nitro-benzene,
1-hydroxy-3-nitro-4-[N-(β-hydroxyethyl)amino]benzene,
1,4-diamino-2-nitrobenzene,
1-amino-2-nitro-4-(methylamino)benzene,
N-(β-hydroxyethyl)-2-nitro-para-phenylenediamine,
1-amino-2-nitro-4-(β-hydroxyethyl)amino-5-chloro-benzene,
2-nitro-4-aminodiphenylamine,
1-amino-3-nitro-6-hydroxybenzene,
1-(β-aminoethyl)amino-2-nitro-4-(β-hydroxyethyl-oxy)benzene,
1-(β,γ-dihydroxypropyl)oxy-3-nitro-4-[(β-hydroxy-ethyl)amino]benzene,
1-hydroxy-3-nitro-4-aminobenzene,
1-hydroxy-2-amino-4,6-dinitrobenzene,
1-methoxy-3-nitro-4-[(β-hydroxyethyl)amino]benzene,
2-nitro-4′-hydroxydiphenylamine,
1-amino-2-nitro-4-hydroxy-5-methylbenzene.

The additional direct dye can also be chosen from yellow and green-yellow nitrobenzene direct dyes. Mention may be made, for example, of the compounds chosen from:
1-β-hydroxyethyloxy-3-methylamino-4-nitrobenzene,
1-methylamino-2-nitro-5-[(β,γ-dihydroxypropyl)oxy]-benzene,
1-(β-hydroxyethyl)amino-2-methoxy-4-nitrobenzene,
1-(β-aminoethyl)amino-2-nitro-5-methoxybenzene,
1,3-di(β-hydroxyethyl)amino-4-nitro-6-chlorobenzene,
1-amino-2-nitro-6-methylbenzene,
1-(β-hydroxyethyl)amino-2-hydroxy-4-nitrobenzene,
N-(β-hydroxyethyl)-2-nitro-4-trifluoromethylaniline,
4-(β-hydroxyethyl)amino-3-nitrobenzenesulfonic acid,
4-ethylamino-3-nitrobenzoic acid,
4-(β-hydroxyethyl)amino-3-nitro-1-chlorobenzene,
4-(β-hydroxyethyl)amino-3-nitro-1-methylbenzene,
4-(β,γ-dihydroxypropyl)amino-3-nitro-1-trifluoro-methylbenzene,
1-(β-ureidoethyl)amino-4-nitrobenzene,
1,3-diamino-4-nitrobenzene,
1-hydroxy-2-amino-5-nitrobenzene,
1-amino-2-[tris(hydroxymethyl)methyl]amino-5-nitro-benzene,
1-(β-hydroxyethyl)amino-2-nitrobenzene,
4-(β-hydroxyethyl)amino-3-nitrobenzamide.

Mention may also be made of blue or purple nitrobenzene direct dyes, such as, for example:
1-(β-hydroxyethyl)amino-4-[N,N-bis(β-hydroxyethyl)-amino]-2-nitrobenzene,
1-(γ-hydroxypropyl)amino-4-[N,N-bis(β-hydroxyethyl)-amino]-2-nitrobenzene,
1-(β-hydroxyethyl)amino-4-[(N-methyl, N-β-hydroxy-ethyl)amino]-2-nitrobenzene,
1-(β-hydroxyethyl)amino-4-[(N-ethyl, N-β-hydroxy-ethyl)amino]-2-nitrobenzene,
1-(β,γ-dihydroxypropyl)amino-4-[(N-ethyl, N-p-hydroxy-ethyl)amino]-2-nitrobenzene,
the 2-nitro-para-phenylenediamines of following formula:

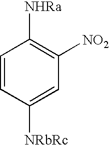

in which:
Rb represents a C₁-C₄ alkyl radical or a β-hydroxyethyl or β-hydroxypropyl or γ-hydroxy-propyl radical;
Ra and Rc, which are identical or different, represent a β-hydroxyethyl, β-hydroxypropyl, γ-hydroxypropyl or β,γ-dihydroxypropyl radical, one at least of the Rb, Rc or Ra radicals representing a γ-hydroxypropyl radical and Rb and Rc not being able to simultaneously denote a β-hydroxyethyl radical when Rb is a γ-hydroxypropyl radical, such as those described in French Patent FR 2 692 572.

Mention may also be made, among the azo direct dyes which can be used according to the invention, of the cationic azo dyes described in patent applications WO 95/15144, WO 95/01772, EP 714 954, FR 2 822 696, FR 2 825 702, FR 2 825 625, FR 2 822 698, FR 2 822 693, FR 2 822 694, FR 2 829 926, FR 2 807 650, WO 02/078660, WO 02/100834, WO 02/100369 and FR 2 844 269. Mention may very particularly be made, among these compounds, of the following dyes:
1,3-dimethyl-2-[[4-(dimethylamino)phenyl]azo]-1H-imidazolium chloride,
1,3-dimethyl-2-[(4-aminophenyl)azo]-1H-imidazolium chloride,
1-methyl-4-[(methylphenylhydrazono)methyl]-pyridinium methyl sulfate.

Mention may also be made, among azo direct dyes, of the following dyes described in the Color Index International, 3rd edition:
Disperse Red 17
Acid Yellow 9
Acid Black 1
Basic Red 22
Basic Red 76
Basic Yellow 57
Basic Brown 16
Acid Yellow 36
Acid Orange 7
Acid Red 33
Acid Red 35
Basic Brown 17
Acid Yellow 23
Acid Orange 24
Disperse Black 9.

Mention may also be made of 1-(4′-aminodiphenylazo)-2-methyl-4-[bis(β-hydroxyethyl)amino]benzene and 4-hydroxy-3-(2-methoxyphenylazo)-1-naphthalenesulfonic acid.

Mention may be made, among quinone direct dyes, of the following dyes:
Disperse Red 15
Solvent Violet 13
Acid Violet 43
Disperse Violet 1
Disperse Violet 4
Disperse Blue 1
Disperse Violet 8
Disperse Blue 3
Disperse Red 11
Acid Blue 62
Disperse Blue 7
Basic Blue 22
Disperse Violet 15
Basic Blue 99
   and of the following compounds:
1-N-methylmorpholiniumpropylamino-4-hydroxy-anthraquinone,
1-aminopropylamino-4-methylaminoanthraquinone,
1-aminopropylaminoanthraquinone,
5-β-hydroxyethyl-1,4-diaminoanthraquinone,
2-aminoethylaminoanthraquinone,
1,4-bis(β,γ-dihydroxypropylamino)anthraquinone.

Mention may be made, among azine dyes, of the following compounds:
Basic Blue 17
Basic Red 2.

Mention may be made, among the triarylmethane dyes which can be used according to the invention, of the following compounds:
Basic Green 1
Acid Blue 9
Basic Violet 3
Basic Violet 14
Basic Blue 7
Acid Violet 49
Basic Blue 26
Acid Blue 7

Mention may be made, among the indoamine dyes which can be used according to the invention, of the following compounds:
2-β-hydroxyethylamino-5-[bis(β-4′-hydroxyethyl)-amino]anilino-1,4-benzoquinone,
2-β-hydroxyethylamino-5-(2′-methoxy-4′-amino)-anilino-1,4-benzoquinone,
3-N-(2′-chloro-4′-hydroxy)phenylacetylamino-6-methoxy-1,4-benzoquinone imine,
3-N-(3′-chloro-4′-methylamino)phenylureido-6-methyl-1,4-benzoquinone imine,
3-[4′-N-(ethyl, carbamylmethyl)amino]phenylureido-6-methyl-1,4-benzoquinone imine.

Mention may be made, among the dyes of tetraazapenta-methine type which can be used according to the invention, of the following compounds which appear in the table below, An being defined as above:

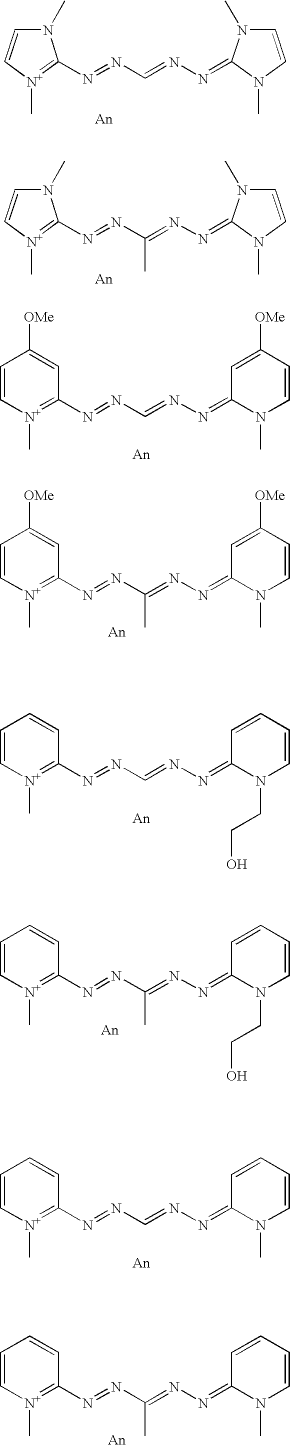

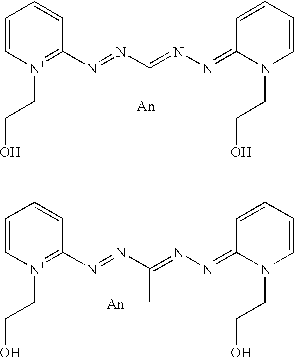

Mention may be made, among the natural direct dyes which can be used according to the invention, of lawsone, juglone, alizarin, purpurin, carminic acid, kermesic acid, purpurogallin, protocatechualdehyde, indigo, isatin, curcumin, spinulosin or apigenidin. Use may also be made of the extracts or decoctions comprising these natural dyes and in particular cataplasms or henna-based extracts.

If they are present, the content of additional direct dye(s) in the composition generally varies from 0.001 to 20% by weight, with respect to the weight of the composition, and preferably from 0.01 to 10% by weight, with respect to the weight of the composition.

The medium appropriate for dyeing, also known as dyeing vehicle, is generally composed of water or of a mixture of water and of at least one organic solvent in order to dissolve the compounds which would not be sufficiently soluble in the water.

More particularly, the organic solvents are chosen from linear or branched, preferably saturated, monoalcohols or diols comprising 2 to 10 carbon atoms, such as ethyl alcohol, isopropyl alcohol, hexylene glycol (2-methyl-2,4-pentanediol), neopentyl glycol and 3-methyl-1,5-pentanediol; aromatic alcohols, such as benzyl alcohol or phenylethyl alcohol; glycols or glycol ethers, such as, for example, ethylene glycol monomethyl, monoethyl and monobutyl ethers, propylene glycol or its ethers, such as, for example, propylene glycol monomethyl ether, butylene glycol or dipropylene glycol; and diethylene glycol alkyl ethers, in particular C₁-C₄ alkyl ethers, such as, for example, diethylene glycol monoethyl ether or monobutyl ether, alone or as a mixture.

The usual solvents described above, if they are present, generally represent from 1 to 40% by weight, preferably from 5 to 30% by weight, with respect to the total weight of the composition.

The dyeing composition in accordance with the invention can also include various adjuvants conventionally used in compositions for dyeing the hair, such as anionic, cationic, nonionic, amphoteric or zwitterionic surface-active agents or their mixtures, anionic, cationic, nonionic, amphoteric or zwitterionic polymers or their mixtures, inorganic or organic thickening agents and in particular anionic, cationic, nonionic and amphoteric polymeric associative thickeners, antioxidants, penetration agents, sequestering agents, fragrances, buffers, dispersing agents, conditioning agents, such as, for example, volatile or nonvolatile and modified or unmodified silicones, film-forming agents, ceramides, preservatives or opacifying agents.

These above adjuvants are generally present in amounts, for each of them, of between 0.01 and 20% by weight, with respect to the weight of the composition.

Of course, a person skilled in the art will take care to choose this or these possible additional compounds in such a way that the advantageous properties intrinsically attached to the oxidation dyeing composition in accordance with the invention are not, or not substantially, detrimentally affected by the envisaged addition or additions.

The pH of the dyeing composition in accordance with the invention is generally between 3 and 12 approximately and preferably between 5 and 11 approximately. It can be adjusted to the desired value using acidifying or basifying agents commonly used in the dyeing of keratinous fibers or alternatively using conventional buffering systems.

Mention may be made, among acidifying agents, by way of example, of inorganic or organic acids, such as hydrochloric acid, orthophosphoric acid, sulfuric acid, carboxylic acids, such as acetic acid, tartaric acid, citric acid or lactic acid, or sulfonic acids.

Mention may be made, among basifying agents, by way of example, of aqueous ammonia, alkaline carbonates, alkanolamines, such as mono-, di- and triethanolamine and their derivatives, sodium hydroxide, potassium hydroxide and the compounds of following formula:

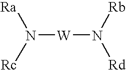

in which W is a propylene residue optionally substituted by a hydroxyl group or a C₁-C₄ alkyl radical and Rₐ, R_{b}, R_{c}, and R_{d}, which are identical or different, represent a hydrogen atom or a C₁-C₄ alkyl or C₁-C₄ hydroxyalkyl radical.

The dyeing composition according to the invention can be provided in various forms, such as in the form of liquids, creams or gels or in any other form appropriate for carrying out dyeing of keratinous fibers and in particular of human hair.

The composition according to the invention can additionally comprise at least one oxidizing agent. Reference is made in this case to a ready-for-use composition.

The term “ready-for-use composition” is understood to mean, within the meaning of the present invention, the composition intended to be applied immediately to keratinous fibers, that is to say that it can be stored as is before use or can result from the mixing at the time of use of two or more compositions.

Said composition can also be obtained by mixing compositions according to the invention with an oxidizing composition.

The oxidizing agent can be any oxidizing agent conventionally used in the field. Thus, it can be chosen from hydrogen peroxide, urea hydrogen peroxide, alkali metal bromates, persalts, such as perborates and persulfates, and enzymes, among which may be mentioned peroxidases, 2-electron oxidoreductases, such as uricases, and 4-electron oxygenases, such as laccases. The use of hydrogen peroxide is particularly preferred.

The content of oxidizing agent is generally between 1 and 40% by weight, with respect to the weight of the ready-for-use composition, preferably between 1 and 20% by weight, with respect to the weight of the ready-for-use composition.

Generally, the oxidizing composition used is an aqueous composition and can be in the form of a solution or also of an emulsion.

Usually, the composition devoid of oxidizing agent is mixed with approximately 0.5 to 10 equivalents by weight of the oxidizing composition.

It should be noted that the pH of the ready-for-use composition is more particularly between 4 and 12, preferably between 7 and 11.5.

The pH of the composition can be adjusted using a basifying or acidifying agent chosen in particular from those mentioned above in the context of the description according to the invention.

Another subject matter of the invention is a coloring process which comprises the application of a dyeing composition according to the invention to dry or wet keratinous fibers.

The application to the fibers of the dyeing composition comprising the compound or compounds of formula (I) or its addition salts with an acid, optionally at least one oxidation base, optionally used in combination with at least one coupler, and optionally at least one additional direct dye can be carried out in the presence of oxidizing agent.

This oxidizing agent can be added to the composition comprising the compound or compounds of formula (I) and the optional oxidation base, couplers and/or additional direct dyes either at the time of use or directly on the keratinous fiber.

The oxidizing composition can also include various adjuvants conventionally used in compositions for dyeing the hair and as defined above.

The pH of the oxidizing composition including the oxidizing agent is such that, after mixing with the dyeing composition, the pH of the resulting composition applied to the keratinous fibers preferably varies between 4 and 12 approximately and more preferably still between 7 and 11.5. It can be adjusted to the desired value using acidifying or basifying agents generally used in the dyeing of keratinous fibers and as defined above.

The composition which is finally applied to the keratinous fibers can be provided in various forms, such as in the form of liquids, creams or gels or in any other form appropriate for carrying out dyeing of keratinous fibers and in particular human hair.

According to a specific embodiment, the composition according to the invention is devoid of oxidation base and of coupler.

The composition applied can optionally comprise at least one oxidizing agent.

The composition is thus applied to dry or wet keratinous fibers and then left for a leave-in time sufficient to obtain the desired coloring.

Whatever the alternative form selected (with or without oxidizing agent), the leave-in time is generally between a few seconds and one hour, preferably between 3 and 30 minutes.

The temperature at which the composition is left to act is generally between 15 and 220° C., more particularly between 15 and 80° C., preferably between 15 and 40° C.

At the end of the leave-in time, the composition is preferably removed by rinsing with water, optionally followed by washing with a shampoo and then optionally by drying.

Another subject matter of the invention is a dyeing kit or multicompartment device in which a first compartment includes the dyeing composition of the invention and a second compartment includes the oxidizing composition. This device can be equipped with a means which makes it possible to deliver the desired mixture to the hair, such as the devices described in patent FR-2 586 913.

The example which follows serves to illustrate the invention without, however, exhibiting a limiting nature.

### EXAMPLE

### 1—Synthesis of compound 4

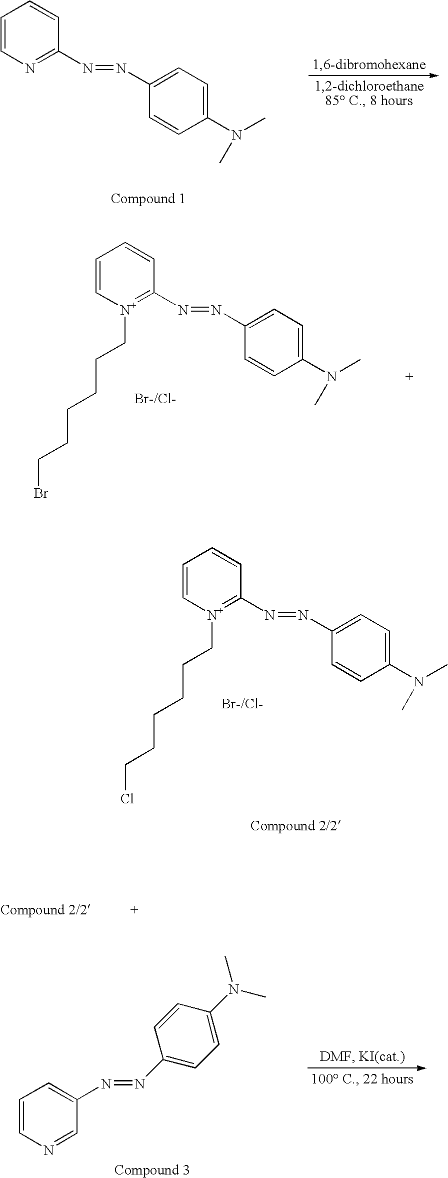

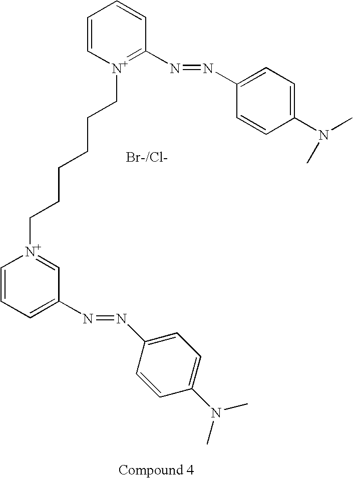

Compound 1 is available commercially (Interchim).

### Stage 1

1.13 g of compound 1 are stirred at 85° C. for 8 hours in the presence of 3.86 ml of 1,6-dibromohexane in 80 ml of 1,2-dichloroethane. The reaction medium is subsequently brought back to ambient temperature. The reaction medium is diluted with dichloromethane and washed with water. The organic phase is separated, dried over sodium sulfate, filtered and concentrated under reduced pressure. A dark purple powder is obtained (compound 2/2′, mixture of brominated compound and chlorinated compound).

The ¹H NMR and mass analyses are in accordance with the expected product (mixture of brominated compound and chlorinated compound).

### Stage 2

0.95 g of compound 2/2′ is stirred at 100° C. for 22 hours in the presence of 0.45 g of compound 3 and of 0.05 g of KI in 15 ml of dimethylformamide. The reaction medium is subsequently brought back to ambient temperature and then poured into diisopropyl ether. The residue obtained is washed with acetone and then dissolved in dichloromethane. The residual insoluble material is subsequently filtered off. The dye in solution in the dichloromethane is precipitated by addition of ethyl acetate. The dark purple precipitate is filtered off, dried under vacuum and then analyzed.

The ¹H NMR and mass analyses are in accordance with the structure of the expected compound 4.

### Synthesis of Compound 9

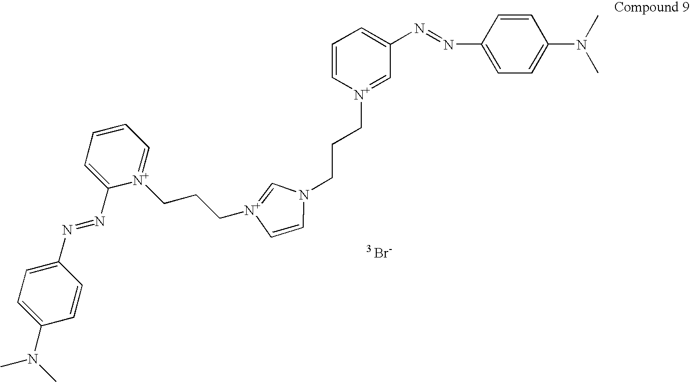

### Stage 1

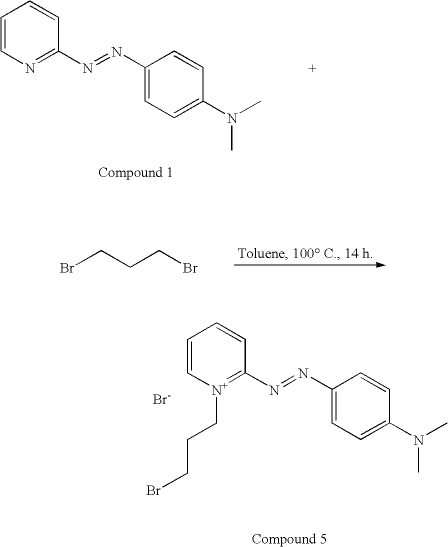

Compound 1 is a commercial product.

Compound 1 (30 g) is stirred at 100° C. for 14 hours in the presence of 68 ml of 1,3-dibromopropane in 350 ml of toluene in a three-necked flask surmounted by a reflux condenser.

After reaction, the reaction medium is cooled to ambient temperature and then poured onto ethyl acetate (500 ml). The precipitate obtained is filtered off, then washed several times with ethyl acetate and finally dried under vacuum. 44 g of a dark purple powder corresponding to compound 5 are obtained.

The analyses are in accordance with the expected product.

### Stage 2

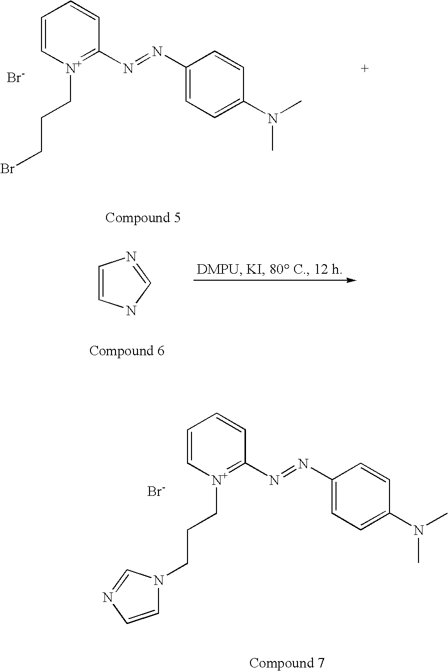

Compound 6 is a commercial product.

Compound 5 (5 g), compound 6 (4.9 g) and potassium iodide (2.4 g) are stirred at 80° C. for 12 hours in 30 ml of 1,3-dimethyl-3,4,5,6-tetrahydro-2(1H)-pyrimidinone (DMPU) in a three-necked flask surmounted by a reflux condenser.

After reaction, the reaction medium is cooled to ambient temperature and then poured onto ethyl acetate (200 ml). The precipitate obtained is filtered off, then washed several times with ethyl acetate and finally dried under vacuum. 4.5 g of a dark purple powder corresponding to compound 7 are obtained.

The analyses are in accordance with the expected product.

### Stage 3

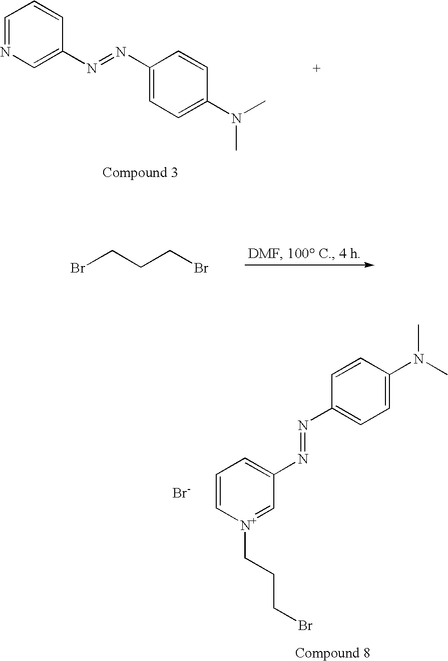

Compound 3 (2.27 g, 0.01 mol) is stirred at 100° C. for 4 hours in the presence of 5.1 ml of 1,3-dibromopropane in 5 ml of DMF in a three-necked flask surmounted by a reflux condenser.

After reaction, the reaction medium is cooled to ambient temperature and then poured onto ethyl acetate (150 ml). The precipitate obtained is filtered off, then washed several times with ethyl acetate and finally dried under vacuum. 3 g of a vivid orange powder corresponding to compound 8 are obtained.

The analyses are in accordance with the expected product.

### Stage 4

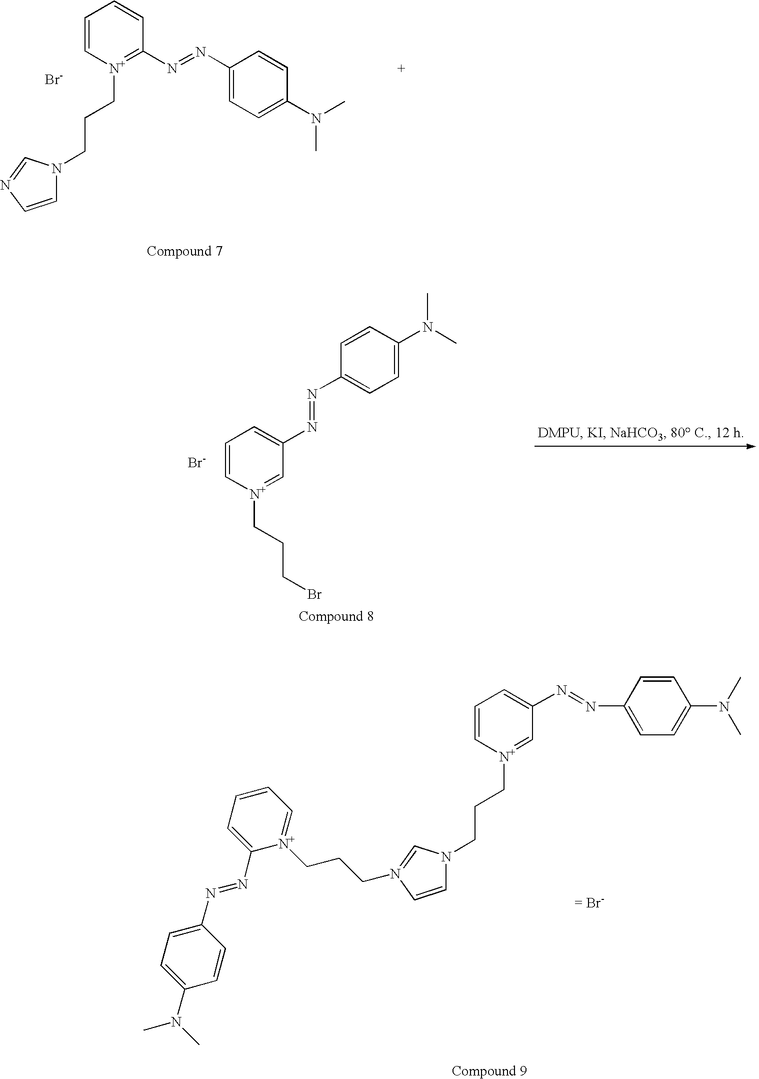

Compound 8 (0.155 g), compound 7 (0.15 g), potassium iodide (36 mg) and sodium hydrogencarbonate (18 mg) are stirred at 80° C. for 12 hours in 4 ml of DMPU in a three-necked flask surmounted by a reflux condenser. After reaction, the reaction medium is cooled to ambient temperature and then poured onto ethyl acetate (15 ml). The precipitate obtained is filtered off, then washed several times with ethyl acetate and finally dried under vacuum. 105 mg of a dull purple powder corresponding to compound 9 are obtained.

The analyses are in accordance with the expected product.

### Dyeing Examples

5×10⁻⁴ mol of compound 4 obtained above is dissolved in 5 ml of a mixture of water (2.5 ml) and of pH 10 buffer (2.5 ml) with the following composition:

2 g of ammonium acetate
40 ml of water

### 20% NH₃ up to pH 9-10

qsp for 100 ml of water

100 g of the above composition are applied to hair at ambient temperature for 30 minutes. The hair is subsequently rinsed with water and dried.

The hair is colored purple-red.

### Comparative Result

Color after Shampooing the Dyes 4 and 5

After 6 Dop shampooing operations at 2%, the lock colored with the symmetrical dye 5 has lost coverage and gloss, in contrast with the lock colored with dye 4 of the invention.

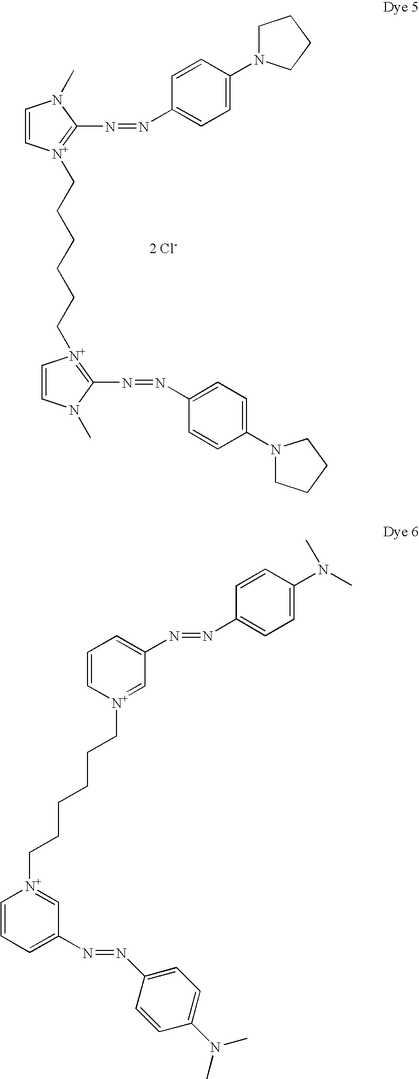

### Change in Dyes 4 and 6 in the Presence of Light

Photodecomposition studies have made it possible to demonstrate that the dyes can decompose relatively significantly toward light. This photodecomposition can result in some cases in the color on the hair being toned down or indeed even vanishing. Studies have shown that dye 6 (symmetrical), in contrast to dye 4 of the invention, results in the color being very significantly toned down after exposure to light.

What is claimed is:

1. A cationic asymmetric diazo compound of following formula (I), its resonance forms and also its addition salts with an acid and/or its solvates: 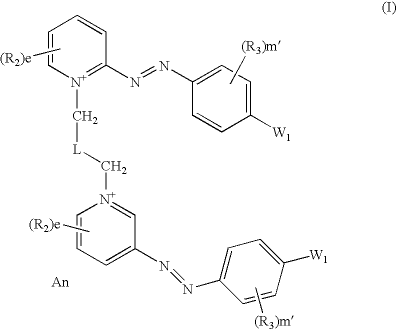
in which formula:
the R₂ radicals, which are identical or different, represent, independently of one another:
an optionally substituted C₁-C₁₆ alkyl radical which is optionally interrupted by one or more heteroatoms and/or by one or more groups comprising at least one heteroatom preferably chosen from oxygen, nitrogen, sulfur, —CO—, —SO₂— or their combinations; said alkyl radical being in addition optionally substituted by one or more identical or different groups chosen from thiol (—SH), C₁-C₄ thioalkyl, (C₁-C₄)alkylsulfinyl or (C₁-C₄)alkylsulfonyl groups;
a hydroxyl group;
a C₁-C₄ alkoxy group;
a C₂-C₄ (poly)hydroxyalkoxy group;
an alkoxycarbonyl (RO—CO—) group in which R represents a C₁-C₄ alkyl radical;
an alkylcarbonyloxy (RCO—O—) radical in which R represents a C₁-C₄ alkyl radical;
an alkylcarbonyl (R—CO—) radical in which R represents a C₁-C₄ alkyl radical;
an amino group;
an amino group substituted by one or two identical or different C₁-C₄ alkyl radicals which optionally carry at least one hydroxyl group, it being possible for the two alkyl radicals optionally to form, with the nitrogen atom to which they are attached, a heterocycle which includes 1 to 3 heteroatoms, preferably 1 or 2 heteroatoms, chosen from N, O or S, preferably N, which comprises from 5 to 7 ring members, which is saturated or unsaturated, which is aromatic or nonaromatic and which is optionally substituted;
an alkylcarbonylamino (RCO—NR′—) group in which the R radical represents a C₁-C₄ alkyl radical and the R′ radical represents a hydrogen or a C₁-C₄ alkyl radical;
an aminocarbonyl ((R)₂N—CO—) group in which the R radicals represent, independently of one another, a hydrogen atom or a C₁-C₄ alkyl radical;
a ureido (N(R)₂—CO—NR′—) group in which the R and R′ radicals represent, independently of one another, a hydrogen atom or a C₁-C₄ alkyl radical;
an aminosulfonyl ((R)₂N—SO₂—) group in which the R radicals represent, independently of one another, a hydrogen atom or a C₁-C₄ alkyl radical;
an alkylsulfonylamino (RSO₂—NR′—) group in which R represents a C₁-C₄ alkyl radical and R′ represents a hydrogen atom or a C₁-C₄ alkyl radical;
an optionally substituted aryl radical;
an optionally substituted aryl(C₁-C₄)alkyl radical;
an alkylsulfinyl (R—SO—) group in which R represents a C₁-C₄ radical;
an alkylsulfonyl (R—SO₂—) group in which R represents a C₁-C₄ radical;
a nitro group;
a cyano group;
a halogen atom, preferably chlorine or fluorine;
a thiol (HS—) group;
an alkylthio (RS—) group in which the R radical represents an optionally substituted C₁-C₄ alkyl radical;
when e is equal to 2, the two R₂ radicals can optionally form, with the carbon atoms to which they are attached, a 5- or 6-membered, preferably 6-membered, aromatic or nonaromatic, secondary ring optionally substituted by one or more identical or different groups chosen from hydroxyl, C₁-C₄ alkyl, C₁-C₄ alkoxy, C₂-C₄ (poly)hydroxyalkoxy, amino or amino substituted by one or two identical or different C₁-C₄ alkyl radicals which optionally carry at least one hydroxyl group;
e is an integer having a value from 0 to 4; when e is less than 4, the unsubstituted carbon atom or atoms of
the heterocycle carry a hydrogen atom;
the R₃ radicals, which are identical or different, represent, independently of one another:
an optionally substituted C₁-C₁₆ alkyl radical which is optionally interrupted by one or more heteroatoms or by one or more groups comprising at least one heteroatom preferably chosen from oxygen, nitrogen, sulfur, —CO—, —SO₂— or their combinations;
a hydroxyl group;
a C₁-C₄ alkoxy group;
a C₂-C₄ (poly)hydroxyalkoxy group;
an alkoxycarbonyl (RO—CO—) group in which R represents a C₁-C₄ alkyl radical;
an alkylcarbonyloxy (RCO—O—) radical in which R represents a C₁-C₄ alkyl radical;
an alkylcarbonyl (R—CO—) radical in which R represents a C₁-C₄ alkyl radical;
an amino group;
an amino group substituted by one or two identical or different C₁-C₄ alkyl radicals which optionally carry at least one hydroxyl group, it being possible for the two alkyl radicals optionally to form, with the nitrogen atom to which they are attached, a heterocycle which includes 1 to 3 heteroatoms, preferably 1 or 2 heteroatoms, chosen from N, O or S, preferably N, which comprises from 5 to 7 ring members, which is saturated or unsaturated, which is aromatic or nonaromatic and which is optionally substituted;
an alkylcarbonylamino (RCO—NR′—) group in which the R radical represents a C₁-C₄ alkyl radical and the R′ radical represents a hydrogen atom or a C₁-C₄ alkyl radical;
an aminocarbonyl ((R)₂N—CO—) group in which the R radicals represent, independently of one another, a hydrogen atom or a C₁-C₄ alkyl radical;
a ureido (N(R)₂—CO—NR′—) group in which the R and R′ radicals represent, independently of one another, a hydrogen atom or a C₁-C₄ alkyl radical;
an aminosulfonyl ((R)₂N—SO₂—) group in which the R radicals represent, independently of one another, a hydrogen atom or a C₁-C₄ alkyl radical;
an alkylsulfonylamino (RSO₂—NR′—) group in which the R and R′ radicals represent, independently of one another, a hydrogen atom or a C₁-C₄ alkyl radical;
a thiol (HS—) group;
an alkylthio (RS—) group in which the R radical represents a C₁-C₄ alkyl radical;
an alkylsulfinyl (R—SO—) group in which R represents a C₁-C₄ alkyl radical;
an alkylsulfonyl (R—SO₂—) group in which R represents a C₁-C₄ alkyl radical;
a nitro group;
a cyano group;
a halogen atom, preferably chlorine or fluorine;
when m′ is greater than or equal to 2, two adjacent R₃ radicals can form, with the carbon atoms to which they are attached, a 6-membered, aromatic or nonaromatic, secondary ring optionally substituted by one or more identical or different groups chosen from the following groups: hydroxyl, C₁-C₄ alkyl, C₁-C₄ alkoxy, C₂-C₄ (poly)hydroxy-alkoxy, C₁-C₄ alkylcarbonylamino, amino or amino substituted by one or two identical or different C₁-C₄ alkyl radicals which optionally carry at least one hydroxyl group;
m′ is an integer having a value from 0 to 4; when m′ is less than 4, then the unsubstituted carbon atom or atoms of the aromatic ring carry a hydrogen atom;
W₁, which are identical or different, represent, independently of one another:
a hydrogen atom;
a halogen atom chosen from bromine, chlorine or fluorine, preferably chlorine and fluorine;
an —NR₅R₆, —OR₇, —NR₄-Ph-NR₅R₆, —NR₄-Ph-OR₇, —O-Ph-OR₇ or —O-Ph-NR₅R₆ group; with:
R₄ and R₇, which are identical or different, representing a hydrogen atom, an optionally substituted C₁-C₂₀, preferably C₁-C₁₆, alkyl radical, an optionally substituted ar(C₁-C₃)alkyl radical or an optionally substituted phenyl radical;
R₅ and R₆, which are identical or different, representing a hydrogen atom, an optionally substituted C₁-C₂₀, preferably C₁-C₁₆, alkyl radical, an optionally substituted phenyl radical, an optionally substituted ar(C₁-C₃)alkyl radical or an alkylcarbonyl (R—CO—) radical in which R is a C₁-C₄ alkyl radical;
R₅ and R₆ being able optionally to form, with the nitrogen atom to which they are attached, a heterocycle which includes 1 to 3 heteroatoms, preferably 1 or 2 heteroatoms, chosen from N, O or S, preferably N, which comprises from 5 to 7 ring members, which is saturated or unsaturated, which is aromatic or nonaromatic and which is optionally substituted;
R₅ and R₆ being able to form, with the carbon atom of the aromatic ring adjacent to that to which —NR₅R₆ is attached, a saturated 5- or 6-membered heterocycle;
Ph representing an optionally substituted phenyl radical;
L is a cationic or noncationic connecting arm;
the electrical neutrality of the compound of formula (I) being provided by one or more identical or different anions An which are cosmetically acceptable.

2. The compound as claimed in the preceding claim, characterized in that the identical or different R₂ radicals represent:
a halogen atom chosen from chlorine or fluorine;
a C₁-C₄ alkyl radical optionally substituted by one or more identical or different radicals chosen from hydroxyl, C₁-C₂ alkoxy, C₂-C₄ (poly)hydroxy-alkoxy, amino, (di)(C₁-C₂)alkylamino, thiol (—SH), (C₁-C₄)alkylsulfinyl, (C₁-C₄)alkylsulfonyl or thio(C₁-C₄)alkyl radicals;
a phenyl radical optionally substituted by one or more radicals chosen from hydroxyl, C₁-C₂ alkoxy, C₂-C₄ (poly)hydroxyalkoxy, amino or (di)(C₁-C₂)alkylamino radicals or a halogen atom, such as chlorine or fluorine;
a C₁-C₄ alkoxy radical;
a (C₁-C₄)alkylsulfonylamino radical;
a C₂-C₄ (poly)hydroxyalkoxy radical;
an amino radical;
a (di)(C₁-C₂)alkylamino radical;
a C₂-C₄ (poly)hydroxyalkylamino radical;
an alkylsulfonylamino (RSO₂N—) radical in which the R radical represents a C₁-C₄ alkyl radical;
an aminosulfonyl ((R)₂NSO₂—) radical in which the R radicals represent, independently of one another, a hydrogen atom or a C₁-C₄ alkyl radical;
an alkylthio (RS—) radical in which the R radical represents a C₁-C₄ alkyl radical;
an alkylsulfinyl (RSO—) radical in which the R radical represents a C₁-C₄ alkyl radical;
an alkylsulfonyl (R—SO₂—) radical in which the R radical represents a C₁-C₄ alkyl radical;
an alkylcarbonylamino (RCONR′—) radical in which the R radical represents a hydrogen atom or a C₁-C₄ alkyl radical and the R′ radical represents a hydrogen atom or a C₁-C₄ alkyl radical.

3. The compound as claimed in either of the preceding claims, characterized in that two R₂ radicals can optionally form, with the carbon atoms to which they are attached, a 6-membered aromatic secondary ring optionally substituted by one or more identical or different groups chosen from hydroxyl, C₁-C₄ alkyl, C₁-C₄ alkoxy, amino or amino substituted by one or two identical or different C₁-C₄ alkyl radicals which optionally carry at least one hydroxyl or methylcarbonylamino group.

4. The compound as claimed in one of the preceding claims, characterized in that the identical or different R₃ radicals represent:
a C₁-C₁₆, preferably C₁-C₈, alkyl radical which is optionally substituted;
a halogen atom, such as chlorine or fluorine;
a hydroxyl group;
a C₁-C₂ alkoxy radical;
a C₂-C₄ (poly)hydroxyalkoxy radical;
an amino radical;
an amino radical substituted by one or two identical or different C₁-C₄ alkyl radicals which optionally carry at least one hydroxyl group or at least one C₁-C₄ alkoxy radical, it being possible for the two alkyl radicals to form, with the nitrogen atom to which they are attached, a heterocycle including 1 to 3 heteroatoms, preferably 1 or 2 heteroatoms, chosen from N, O or S, preferably N, the heterocycle comprising from 5 to 7 ring members, being saturated or unsaturated, being aromatic or nonaromatic and optionally being substituted;
an alkylcarbonylamino (RCO—NR′—) radical in which the R radical represents a C₁-C₄ alkyl radical and the R′ radical represents a hydrogen or a C₁-C₄ alkyl radical;
an alkylsulfonylamino (R′SO₂—NR—) radical in which the R radical represents a hydrogen atom or a C₁-C₄ alkyl radical and the R′ radical represents a C₁-C₄ alkyl radical;
an aminosulfonyl ((R)₂N—SO₂—) radical in which the R radicals, which are identical or different, represent a hydrogen atom or a C₁-C₄ alkyl radical;
an alkylthio (RS—) radical in which the R radical represents a C₁-C₄ alkyl radical;
an alkylsulfonyl (R—SO₂—) radical in which the R radical represents a C₁-C₄ alkyl radical.

5. The compound as claimed in one of the preceding claims, characterized in that the identical or different R₃ radicals represent:
a C₁-C₄ alkyl radical optionally substituted by one or more identical or different radicals chosen from the following radicals: hydroxyl, C₁-C₂ alkyl-carbonylamino or amino substituted by two identical or different C₁-C₂ alkyl radicals which optionally carry at least one hydroxyl group or a C₁-C₂ alkoxy radical; these two alkyl radicals can optionally form, with the nitrogen atom to which they are attached, a saturated or unsaturated, optionally aromatic, 5- or 6-membered heterocycle preferably chosen from pyrrolidine, piperazine, homopiperazine, pyrrole, imidazole or pyrazole;
a C₂-C₄ hydroxyalkoxy radical;
a halogen chosen from chlorine or fluorine;
an amino radical;
an amino radical substituted by one or two identical or different C₁-C₂ alkyl radicals which optionally carry at least one hydroxyl group;
a methylcarbonylamino radical;
a methylsulfonylamino radical;
a hydroxyl radical;
a C₁-C₂ alkoxy radical;
a methylsulfonyl radical.

6. The compound as claimed in one of the preceding claims, characterized in that, when the coefficient m′ is greater than or equal to 2, then two adjacent R₃ radicals can form, with the carbon atoms to which they are attached, a 6-membered aromatic secondary ring optionally substituted by one or more identical or different groups chosen from the following groups: hydroxyl, —NR₄-Ph, —NR₄-Ph-NR₅R₆, —NR₄-Ph-OR₇, C₁-C₄ alkyl, C₁-C₄ alkoxy, C₂-C₄ (poly)hydroxyalkoxy, C₁-C₄ alkylcarbonylamino, amino or amino substituted by one or two identical or different C₁-C₄ alkyl radicals which optionally carry at least one hydroxyl group.

7. The compound as claimed in one of the preceding claims, characterized in that two adjacent R₃ radicals can form, with the carbon atoms to which they are attached, a 6-membered aromatic secondary ring optionally substituted by one or more identical or different groups chosen from the hydroxyl, methoxy, ethoxy, 2-hydroxyethyloxy, amino, methylcarbonylamino, (di)(2-hydroxyethyl)amino, —NH-Ph, —NH-Ph-NH₂, —NH-Ph-NHCOCH₃, —NH-Ph-OH or —NH-Ph-OCH₃ groups.

8. The compound as claimed in the preceding claim, characterized in that R₄ and R₇ represent, independently of one another:
a hydrogen atom;
a C₁-C₆ alkyl radical which is optionally substituted, preferably by at least one hydroxyl or C₁-C₂ alkoxy group;
an aryl or arylalkyl radical, such as phenyl or benzyl, the aryl part optionally being substituted by one or more identical or different radicals chosen from chlorine, amino, hydroxyl, C₁-C₂ alkoxy or amino mono- or disubstituted by one or two identical or different C₁-C₄ alkyl radicals which optionally carry at least one hydroxyl group.

9. The compound as claimed in one of the preceding claims, characterized in that the identical or different R₅ and R₆ radicals represent:
a hydrogen atom;
an alkylcarbonyl (R—CO—) radical in which R represents an optionally substituted C₁-C₄ alkyl radical;
a C₁-C₆ alkyl radical which is optionally substituted, preferably by one or more identical or different groups chosen from hydroxyl, C₁-C₂ alkoxy, amino or (di)(C₁-C₄)alkylamino; the alkyl radical can, in addition, be substituted by at least one C₁-C₄ alkylsulfonyl group, at least one C₁-C₄ alkylsulfinyl group or at least one C₁-C₄ alkylcarbonyl group;
an aryl or arylalkyl radical, such as phenyl or benzyl, the aryl part optionally being substituted by one or more identical or different groups chosen from chlorine, amino, hydroxyl, C₁-C₄ alkoxy or amino mono- or disubstituted by one or two identical or different C₁-C₄ alkyl radicals which optionally carry at least one hydroxyl group.

10. The compound as claimed in one of the preceding claims, characterized in that the identical or different R₅ and R₆ radicals represent:
a hydrogen atom;
a methylcarbonyl, ethylcarbonyl or propylcarbonyl radical;
an optionally substituted C₁-C₃ alkyl radical, such as methyl, ethyl, 2-hydroxyethyl or 2-methoxy-ethyl;
a phenyl radical optionally substituted by one or more radicals chosen from the following radicals: hydroxyl, C₁-C₂ alkoxy, amino or amino substituted by one or more C₁-C₄ groups which optionally carry at least one hydroxyl group.

11. The compound as claimed in one of claims 1 to 9, characterized in that the R₅ and R₆ radicals form, together with the nitrogen atom to which they are attached, a heterocycle which includes 1 to 3 hetero-atoms, preferably 1 or 2 heteroatoms, chosen from N, O or S, preferably N, which comprises from 5 to 7 ring members, which is saturated or unsaturated, which is aromatic or nonaromatic and which is optionally substituted.

12. The compound as claimed in the preceding claim, characterized in that the heterocycle which comprises from 5 to 7 ring members is chosen from the following heterocycles: piperidine, 2-(2-hydroxyethyl)piperidine, 4-(aminomethyl)piperidine, 4-(2-hydroxyethyl)-piperidine, 4-(dimethylamino)piperidine, piperazine, 1-methylpiperazine, 1-(2-hydroxyethyl)piperazine, 1-(2-aminoethyl)piperazine, 1-hydroxyethylethoxy piperazine, homopiperazine, 1-methyl-1,4-perhydrodiazepine, pyrrole, 1,4-dimethylpyrrole, 1-methyl-4-ethylpyrrole or 1-methyl-4-propylpyrrole.

13. The compound as claimed in one of claims 1 to 9, characterized in that the R₅ and R₆ radicals form, with the carbon atom of the aromatic ring, optionally substituted by a hydroxyl, adjacent to that to which —NR₅R₆ is attached, a saturated 5- or 6-membered heterocycle.

14. The compound as claimed in one of the preceding claims, characterized in that L is a noncationic connecting arm represented by:
a covalent bond;
a C₁-C₄₀, preferably C₁-C₂₀, alkyl radical which is optionally substituted and optionally interrupted by a saturated or unsaturated, aromatic or nonaromatic, 3- to 7-membered (hetero)cycle which is optionally substituted and optionally condensed; said alkyl radical optionally being interrupted by one or more heteroatoms or groups comprising at least one heteroatom, preferably oxygen, nitrogen, sulfur, —CO—, —SO₂— or their combinations; the connecting arm L not comprising an azo, nitro, nitroso or peroxo bond;
an optionally substituted phenyl radical.

15. The compound as claimed in one of claims 1 to 13, characterized in that L is a cationic connecting arm represented by a C₂-C₄₀ alkyl radical carrying at least one cationic charge, which alkyl radical is optionally substituted and/or optionally interrupted by one or more identical or different, saturated or unsaturated, aromatic or nonaromatic, 3- to 7-membered (hetero)cycles and/or optionally interrupted by one or more heteroatoms or groups comprising at least one heteroatom or their combinations, such as, for example, oxygen, nitrogen, sulfur, a —CO— or —SO₂— group or their combinations; the connecting arm L not comprising an azo, nitro, nitroso or peroxo bond; it being understood that the connecting arm L carries at least one cationic charge.

16. The compound as claimed in any one of the preceding claims, characterized in that the anion An represents an organic or inorganic anion or a mixture of organic or inorganic anions which makes it possible to balance the charge or charges of the compounds of formula (I) chosen from a halide, such as chloride, bromide, fluoride or iodide; a hydroxide; a sulfate, a hydrogensulfate; an alkyl sulfate for which the alkyl part, which is linear or branched, is a C₁-C₆ part, such as the methyl sulfate or ethyl sulfate ion; carbonates and hydrogencarbonates; salts of carboxylic acids, such as formate, acetate, citrate, tartrate or oxalate; alkylsulfonates for which the alkyl part, which is linear or branched, is a C₁-C₆ part, such as the methylsulfonate ion; arylsulfonates for which the aryl part, which is preferably phenyl, is optionally substituted by one or more C₁-C₄ alkyl radicals, such as, for example, 4-toluoylsulfonate; alkylsulfonyls, such as mesylate.

17. The compound as claimed in any one of the preceding claims, characterized in that it corresponds to the following formulae (I′), (I″) or (I′″), and also their resonance forms and/or their addition salts with an acid and/or their solvates: 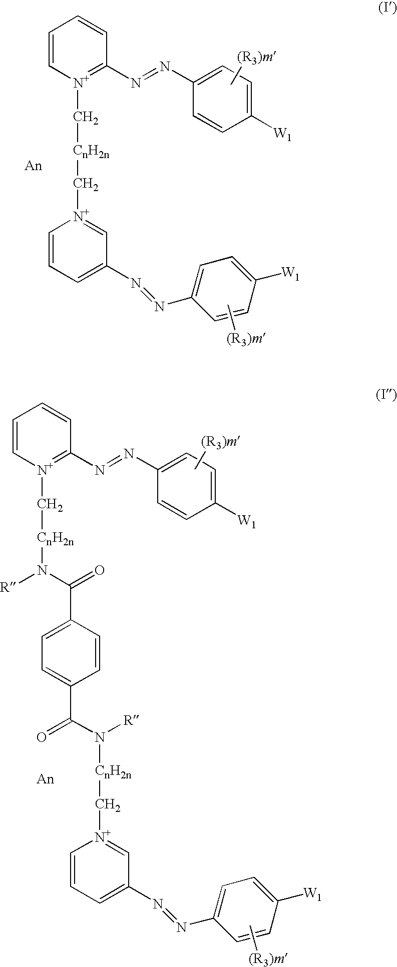 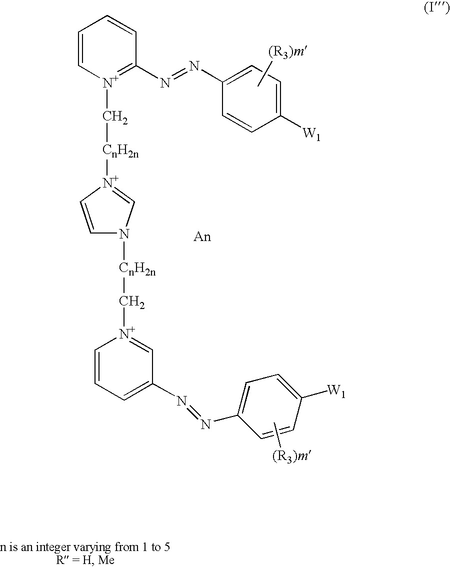
in which R₂, n′ and An have the same meanings as above.

18. The compound as claimed in any one of the preceding claims, characterized in that it corresponds to the following formulae, to their addition salts with an acid or their solvates:

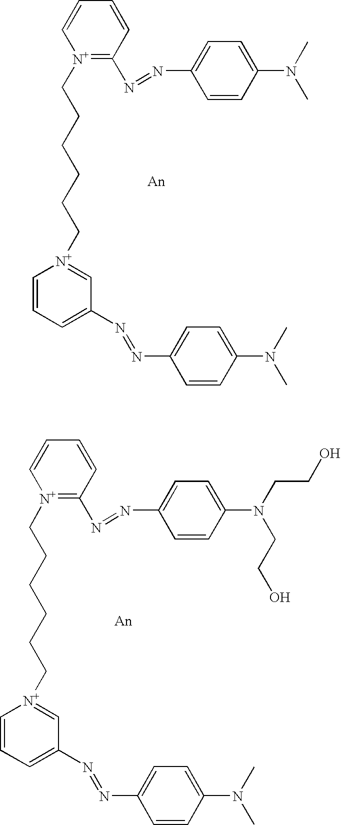

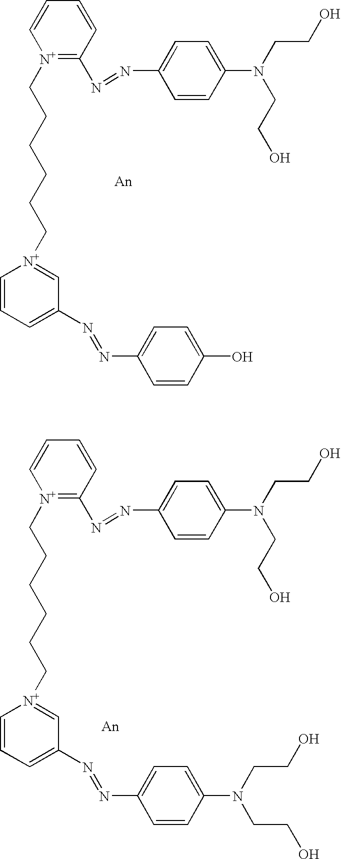

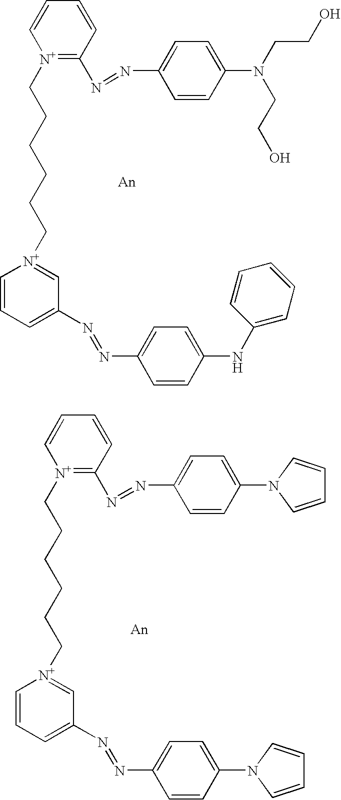

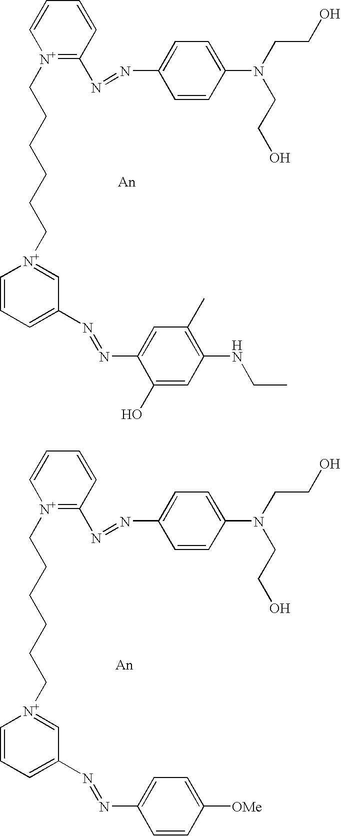

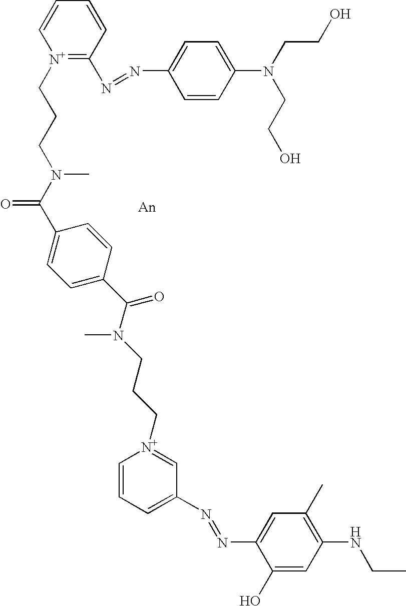

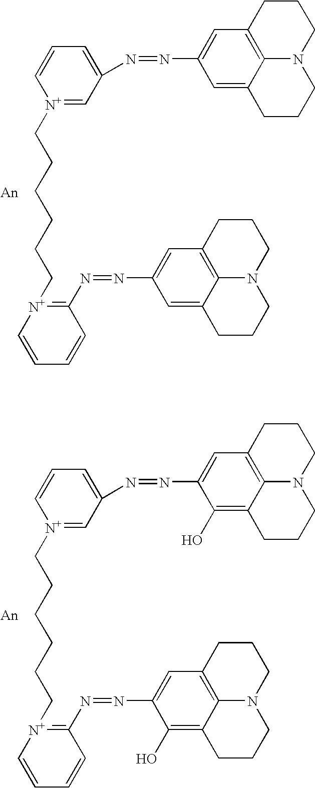

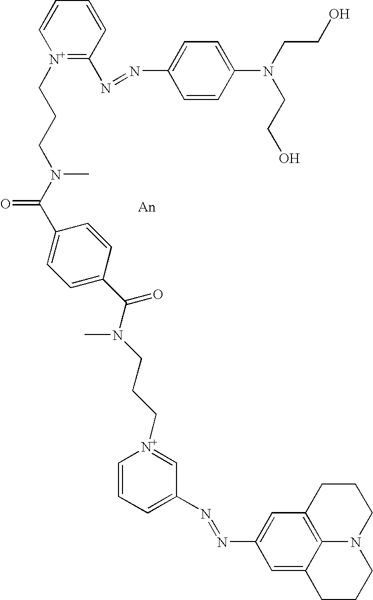

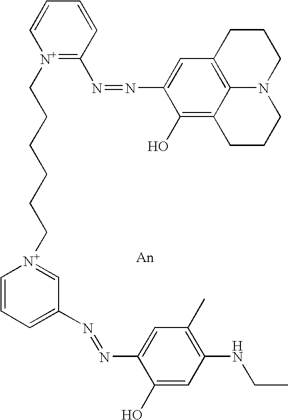

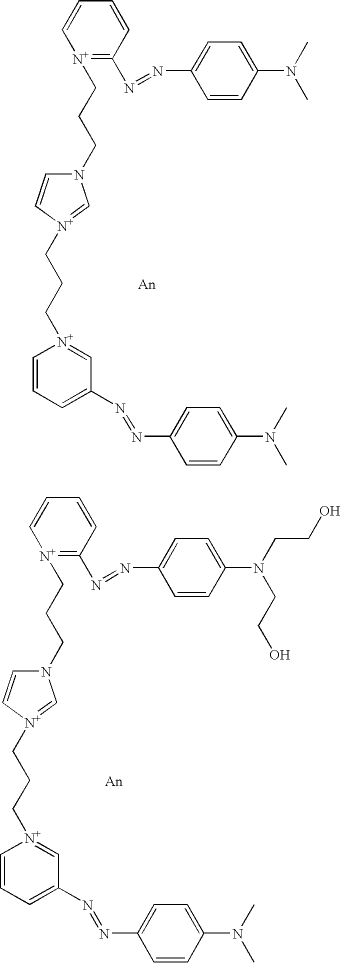

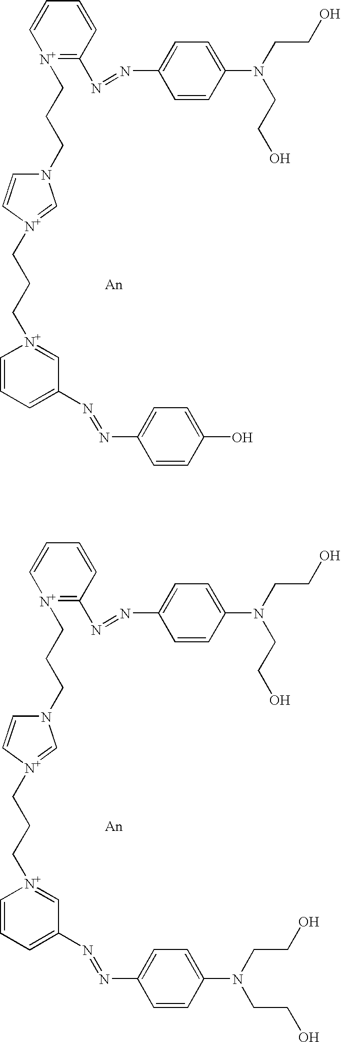

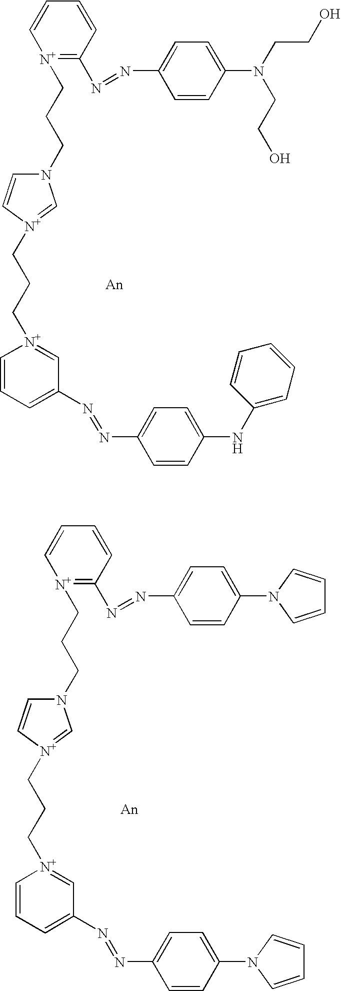

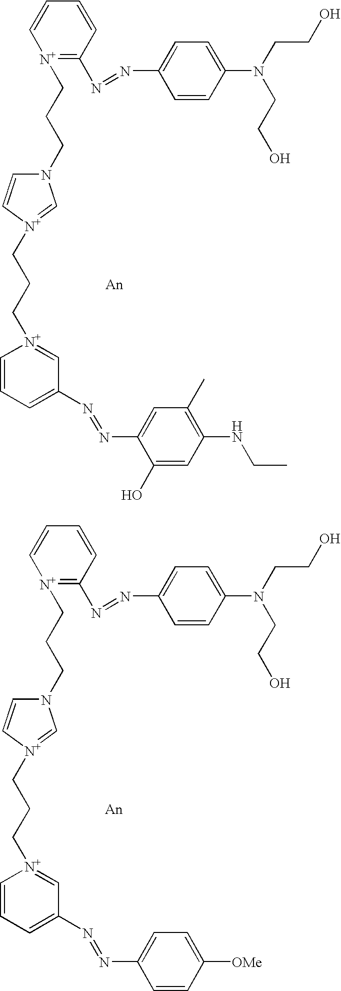

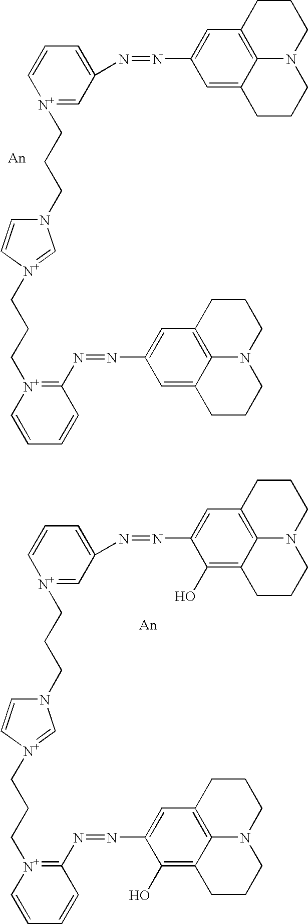

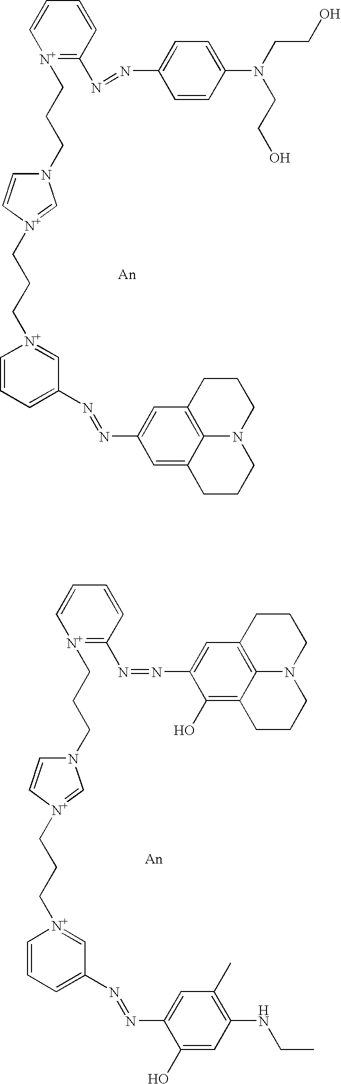

19. A dyeing composition comprising, in a medium appropriate for the dyeing of keratinous fibers, as direct dye, at least one compound of formula (I), its addition salts with an acid and/or its solvates as claimed in any one of the preceding claims.

20. The composition as claimed in the preceding claim, characterized in that the content of compound of formula (I) or of each of the compounds of formula (I) varies between 0.001 and 20% by weight, with respect to the total weight of the dyeing composition, more particularly between 0.01 and 10% by weight.

21. The composition as claimed in either one of claims 19 and 20, characterized in that it comprises at least one additional direct dye, at least one oxidation base, optionally in combination with at least one coupler.

22. The composition as claimed in the preceding claim, characterized in that the additional direct dye is a cationic or nonionic dye chosen from nitrobenzene dyes, azo, azomethine, methine, tetraazapentamethine, anthraquinone, naphthoquinone, benzoquinone, phenothiazine, indigoid, xanthene, phenanthridine or phthalocyanine dyes, those derived from triarylmethane or natural dyes, alone or as mixtures.

23. The composition as claimed in claim 21, characterized in that the oxidation base is chosen from p-phenylenediamines, bisphenylalkylenediamines, o-aminophenols, p-aminophenols or heterocyclic bases.

24. The composition as claimed in claim 21, characterized in that the coupler is chosen from m-aminophenols, m-phenylenediamines, m-diphenols, naphthols, heterocyclic couplers, their addition salts with an acid and their mixtures.

25. The composition as claimed in any one of claims 19 to 24, characterized in that it comprises at least one oxidizing agent.

26. A process for coloring keratinous fibers which consists in bringing a composition as claimed in any one of claims 19 to 25 into contact with said dry or wet fibers for a period of time sufficient to obtain the desired effect.

27. A multicompartment device in which the first compartment includes the as claimed in any one of claims 19 to 24 and a second compartment includes an oxidizing composition.
